(19) United States
(12) Patent Application Publication
(10) Pub. No.: US 2024/0285816 A1
(43) Pub. Date: Aug. 29, 2024

(54) FORMULATIONS FOR RADIOTHERAPY AND DIAGNOSTIC IMAGING

(71) Applicant: Clarity Pharmaceuticals Ltd, Eveleigh (AU)

(72) Inventors: Matthew John HARRIS, Hunters Hill (AU); Ellen Marianne Van Dam, Canterbury (AU); Charmaine Marie Jeffery, Dutton Park (AU)

(21) Appl. No.: 18/409,431

(22) Filed: Jan. 10, 2024

Related U.S. Application Data
(63) Continuation of application No. 17/164,338, filed on Feb. 1, 2021, now abandoned.
Application No. 17/164,338 is a continuation of application No. 16/347,272, filed on May 3, 2019, now abandoned.

(30) Foreign Application Priority Data
Nov. 4, 2016 (AU) 2016904515

(51) Int. Cl. A61K 51/04; A61K 51/02; A61K 51/08

(52) U.S. Cl. CPC A61K 51/0485; A61K 51/025; A61K 51/0497; A61K 51/088

(57) ABSTRACT

The present invention relates to formulations of radiolabelled compounds that are of use in radiotherapy and diagnostic imaging.

### CROSS-REFERENCE TO RELATED APPLICATIONS

This application is a continuation of U.S. application Ser. No. 17/164,338, filed Feb. 1, 2021, which is a continuation of U.S. application Ser. No. 16/347,272, filed May 3, 2019, which is a 35 U.S.C. 371 national stage filing of International Application No. PCT/AU2017/051205, filed Nov. 2, 2017, which claims priority to Australian Patent Application 2016904515, filed Nov. 4, 2016. The contents of the aforementioned applications are hereby incorporated by reference in their entirety.

### FIELD

The present invention relates to formulations of radiolabelled compounds that are of use in radiotherapy and diagnostic imaging.

### BACKGROUND

Radiolabelled compounds or ligands may be used as radiopharmaceuticals in applications such as radiotherapy or diagnostic imaging. Of particular use, are radiolabelled compounds that show some propensity for selectively targeting a particular site in vivo, (for example, a particular receptor), and subsequently delivering the radioisotope to the desired site of action. This requires that the ligand comprises a component to complex the radioisotope and a further component to target the desired site.

One of the known problems associated with such a ligand is the premature dissociation of the radioisotope prior to the arrival of the ligand-radioisotope complex at the site of action. Not only does this reduce the efficacy of the complex, but the loss of the radioisotope to areas where radiotherapeutic effects are not intended, may result in adverse consequences.

Dissociation of the radioisotope from the ligand may occur as a result of transchelation, where the radioisotope transfers to another biological ligand in vivo. Again, this leads to a reduced therapeutic effect and also delivery of a radioisotope to areas where treatment is not required.

The ligand to be radiolabelled and the radioisotope are usually stored and transported to the patient in separate containers to minimise the above problems relating to dissociation prior to administration. The ligand may be transported as a lyophilized powder at reduced temperatures in order to prolong stability of the compound. The radioisotope can then be combined with the ligand to form the radiopharmaceutical, just prior to administration, which can serve to minimise dissociation of the radioisotope prior to the complex reaching the site of action.

Another problem associated with radiolabelled compounds is that the use of a radioisotope may result in radiolysis, or destruction of the ligand. As a radioisotope undergoes spontaneous decay and subsequent release of radiation, this energy may be sufficient to induce cleavage of bonds and cause subsequent destruction of the ligand. In addition to the reduced efficacy of the radiopharmaceutical, release of the radioisotope also occurs, resulting in the delivery of radiation to unwanted sites.

As many radiopharmaceuticals are designed to be administered parenterally, i.e. non-orally and usually as a solution, the ligand itself must be soluble in a pharmaceutically acceptable solvent or carrier. As is known in the art, the solubility of a particular compound in any given solvent may be unpredictable. Although the solubility of a particular compound in a particular solvent may be known, the solubility of an analogue of the compound in a different solvent system may be quite different. This then presents difficulties to one seeking to develop a formulation of a compound and especially a pharmaceutically acceptable injectable formulation.

Pharmaceutical formulations typically include one or more excipients that affect the compound in some way, such as the enhancement of solubility of the compound or increasing stability of the compound while in solution. Alternatively, additional excipients may be used to provide other features to the formulation, such as preservatives, buffers and the like.

While many thousands of formulations of ligand-radioisotope complexes have been documented, there is no expectation that the excipients used in such formulations would provide the required solubility and bioavailability of any newly developed complex. Furthermore, one cannot expect that a particular combination of excipients would further prevent or minimise the dissociation of the radioisotope or minimise radiolysis from occurring.

Accordingly, desirable formulations of ligand-radioisotope complexes need to be tailored in order to display the requisite stability in relation to radiolysis and dissociation of the radioisotope, while also being pharmaceutically acceptable. The present invention seeks to address these problems in relation to a specific ligand complex.

### SUMMARY

In one aspect of the present invention, there is provided an aqueous formulation for parenteral administration comprising a compound of Formula (I), or a salt thereof, complexed with a Cu ion:

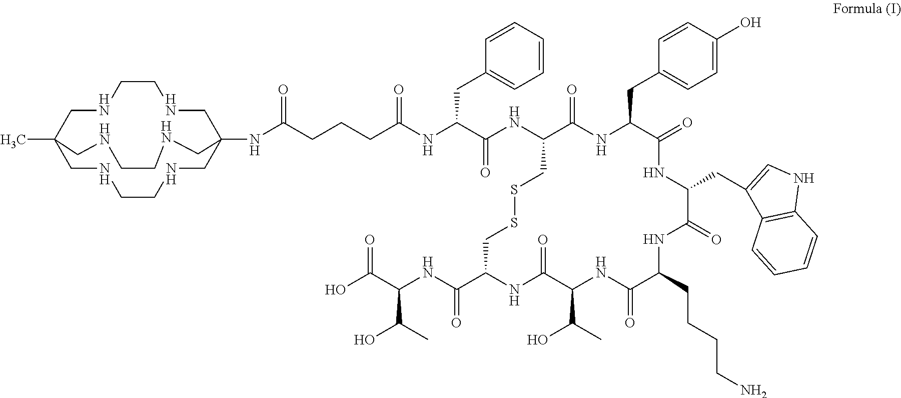 [SMILES: CC(O)[C@H](NC(=O)[C@@H]1CSSC[C@H](NC(=O)[C@@H](Cc2ccccc2)NC(=O)CCCC(=O)NC23CNCCNCC(C)(CNCCNC2)CNCCNC3)C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N1)C(=O)O]

the formulation further comprising:
about 7 to about 13% (v/v) ethanol;
about 0.3 to about 1.2% (w/v) sodium chloride;
about 0.02 to about 0.1% (w/v) gentisic acid or a salt thereof;
   wherein the formulation has a pH of between about 4 to about 8.

In another aspect of the present invention, there is provided an aqueous formulation for parenteral administration comprising a compound of Formula (I), or a salt thereof, complexed with a Cu ion:

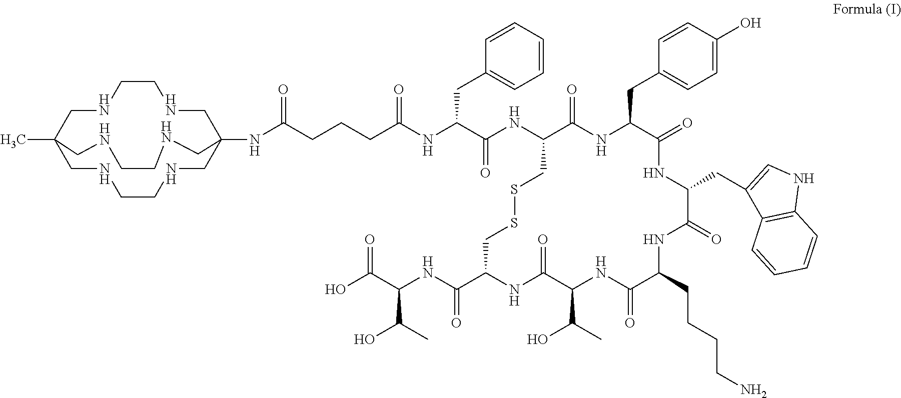 [SMILES: CC(O)[C@H](NC(=O)[C@@H]1CSSC[C@H](NC(=O)[C@@H](Cc2ccccc2)NC(=O)CCCC(=O)NC23CNCCNCC(C)(CNCCNC2)CNCCNC3)C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N1)C(=O)O]

the formulation further comprising:
about 7 to about 13% (v/v) ethanol;
about 0.3 to about 1.2% (w/v) sodium chloride;
about 0.02 to about 0.1% (w/v) gentisic acid or a salt thereof; and
about 1.0 to about 4.0 mg/mL L-methionine or a salt thereof;
   wherein the formulation has a pH of between about 4 to about 8.

In an embodiment and in relation to the above two aspects, the compound of Formula (I) is provided as the acetate salt.

According to a further aspect of the present invention, there is provided a process for preparing an aqueous formulation comprising a compound of Formula (I) complexed with a Cu ion, the method comprising the steps of:
i) preparing a buffering solution of an acetate salt, wherein the buffering solution further comprises ethanol and gentisic acid or a salt thereof;
ii) dissolving a compound of Formula (I), or a salt thereof, in the buffering solution obtained from step i);
iii) adding a solution of a Cu ion to the solution obtained from step ii);
iv) filtering the solution obtained from step iii) on to a stationary phase; and
v) washing the stationary phase of step iv) with ethanol and saline;
   to recover an aqueous formulation comprising a compound of Formula (I), or a salt thereof, complexed with a Cu ion.

According to a further aspect of the present invention, there is provided a process for preparing an aqueous formulation comprising a compound of Formula (I) complexed with a Cu ion, the method comprising the steps of:
i) preparing a buffering solution of an acetate salt, wherein the buffering solution further comprises ethanol and gentisic acid or a salt thereof;
ii) dissolving a compound of Formula (I), or a salt thereof, in the buffering solution obtained from step i);
iii) adding a solution of a Cu ion to the solution obtained from step ii);
iv) filtering the solution obtained from step iii) on to a stationary phase; and
v) washing the stationary phase of step iv) with ethanol and saline into a vial containing a solution of L-methionine or a salt thereof;
   to recover an aqueous formulation comprising a compound of Formula (I), or a salt thereof, complexed with a Cu ion.

According to another aspect of the present invention, there is provided an aqueous formulation prepared by a process as defined in an earlier aspect.

The aqueous formulation of the present invention may also be prepared by providing certain components of the formulation as a kit of parts, where the kit comprises at least a compound of Formula (I), or a salt thereof, and the Cu ion that is intended to be complexed with the compound of Formula (I), in which the compound of Formula (I), or a salt thereof, and the Cu ion are provided separately in the kit and may be combined to form the aforementioned complex prior to administration.

Accordingly, in another aspect the present invention provides a kit for making an aqueous formulation for parenteral administration comprising a compound of Formula (I), or a salt thereof, complexed with a Cu ion, the kit comprising:
a container comprising a lyophilised compound of Formula (I)

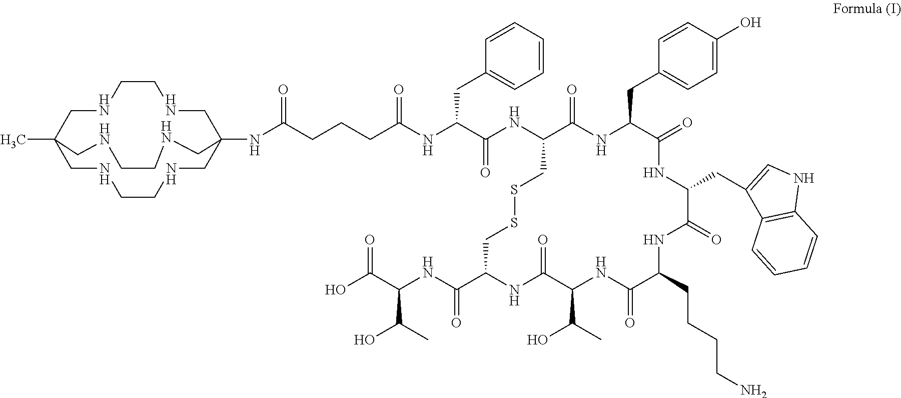 [SMILES: CC(O)[C@H](NC(=O)[C@@H]1CSSC[C@H](NC(=O)[C@@H](Cc2ccccc2)NC(=O)CCCC(=O)NC23CNCCNCC(C)(CNCCNC2)CNCCNC3)C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N1)C(=O)O]

or a salt thereof;
a container comprising a solution of a Cu ion; and
instructions for preparing an aqueous formulation as defined in an earlier aspect, including the addition of a buffered solution of ethanol, sodium chloride and gentisic acid, or a salt thereof.

In another aspect the present invention provides a kit for making an aqueous formulation for parenteral administration comprising a compound of Formula (I), or a salt thereof, complexed with a Cu ion, the kit comprising:
a container comprising a lyophilised compound of Formula (I)

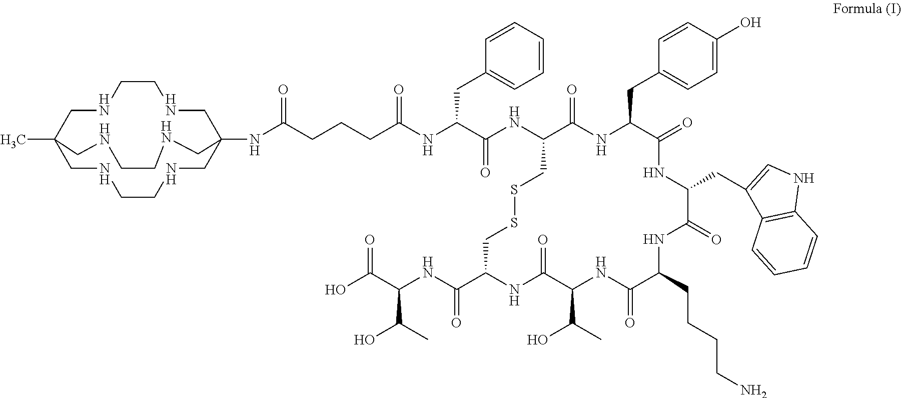 [SMILES: CC(O)[C@H](NC(=O)[C@@H]1CSSC[C@H](NC(=O)[C@@H](Cc2ccccc2)NC(=O)CCCC(=O)NC23CNCCNCC(C)(CNCCNC2)CNCCNC3)C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N1)C(=O)O]

or a salt thereof;
a container comprising a solution of a Cu ion; and
instructions for preparing an aqueous formulation as aforementioned defined, including the addition of a buffered solution of ethanol, sodium chloride, gentisic acid or a salt thereof, and L-methionine or a salt thereof.

A further aspect of the present invention provides a kit for making an aqueous formulation as defined in an earlier aspect for parenteral administration, the kit comprising:
a container comprising a lyophilised compound of Formula (I), or a salt thereof;

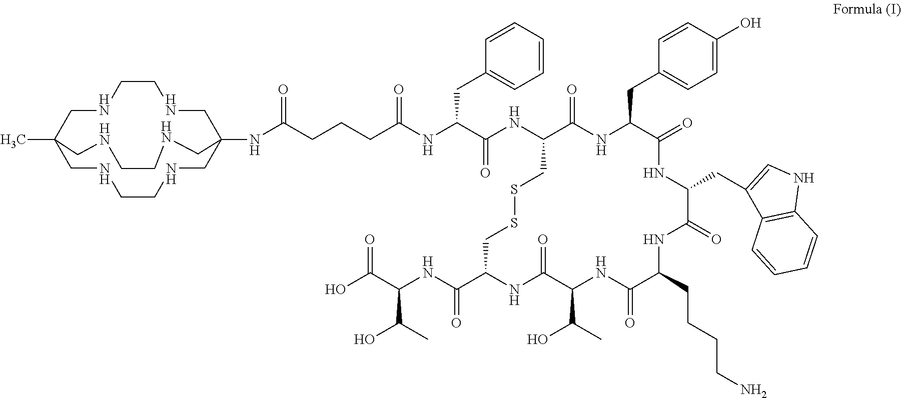 [SMILES: CC(O)[C@H](NC(=O)[C@@H]1CSSC[C@H](NC(=O)[C@@H](Cc2ccccc2)NC(=O)CCCC(=O)NC23CNCCNCC(C)(CNCCNC2)CNCCNC3)C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N1)C(=O)O]

a container comprising a solution of a Cu ion;
a container comprising a buffered solution of ethanol, sodium chloride and gentisic acid, or a salt thereof; and
instructions for preparing an aqueous formulation as defined in an earlier aspect.

A further aspect of the present invention provides a kit for making an aqueous formulation as aforementioned for parenteral administration, the kit comprising:
a container comprising a lyophilised compound of Formula (I), or a salt thereof;

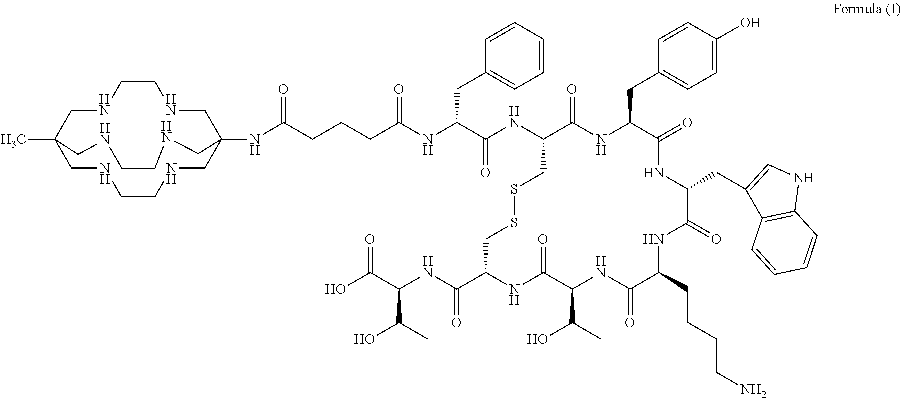 [SMILES: CC(O)[C@H](NC(=O)[C@@H]1CSSC[C@H](NC(=O)[C@@H](Cc2ccccc2)NC(=O)CCCC(=O)NC23CNCCNCC(C)(CNCCNC2)CNCCNC3)C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N1)C(=O)O]

a container comprising a solution of a Cu ion;
a container comprising a buffered solution of ethanol, sodium chloride, gentisic
acid or a salt thereof, and L-methionine or a salt thereof; and
   instructions for preparing an aqueous formulation as defined in an earlier aspect.

Another aspect of the present invention provides a method for radioimaging, diagnosing or treating a cancer, the method comprising administering to a subject in need thereof an aqueous formulation as defined in an earlier aspect.

### BRIEF DESCRIPTION OF THE FIGURES

FIG. 1: Area percent report, using gamma scintillation detector—High performance liquid chromatograph (HPLC) analysis of a low-dose ⁶⁴Cu-SARTATE formulation of Example 1 immediately after preparation (radiochemical yield=606 MBq) representing 97.3% of ⁶⁴Cu detected being present as ⁶⁴Cu-SARTATE.

FIG. 2: Graph of repeat HPLC analyses of low-dose ⁶⁴Cu-SARTATE formulation of Example 1 over 24 hours, using gamma scintillation detector, representing that the radiochemical purity of ⁶⁴Cu-SARTATE remains stable (>90%) over time.

FIG. 3: Area percent report, using gamma scintillation detector—HPLC analysis of a high-dose ⁶⁴Cu-SARTATE formulation of Example 2 immediately after preparation (radiochemical yield=3500 MBq) representing 98.2% of ⁶⁴Cu detected being present as ⁶⁴Cu-SARTATE.

FIG. 4: Graph of repeat HPLC analyses of high-dose ⁶⁴Cu-SARTATE formulation of Example 2 over 45 hours, using gamma scintillation detector, representing that the radiochemical purity of ⁶⁴Cu-SARTATE remains stable (>90%) over time.

FIG. 5: Area percent report, using gamma scintillation detector—HPLC analysis of ⁶⁷Cu-SARTATE formulation of Example 3 immediately after preparation (radiochemical yield=3922 MBq) representing 98.6% of ⁶⁷Cu detected being present as ⁶⁷Cu-SARTATE.

FIG. 6: Graph of repeat HPLC analyses of ⁶⁷Cu-SARTATE formulation of Example 3 over 11 hours, using gamma scintillation detector, representing that the radiochemical purity of ⁶⁷Cu-SARTATE remains stable (>90%) over time.

FIG. 7: Graph of repeat HPLC analyses of ⁶⁴Cu-SARTATE formulation of Example 2 over 43 hours, after incubation in fresh human serum.

FIG. 8: In vitro internalization of ⁶⁴Cu-SARTATE in the SSTR2 over-expressing cell line A427-7 (closed symbols) and with an excess of Tyr³-octreotate (open symbols), for increasing periods of incubation.

FIG. 9: Cell-surface binding of ⁶⁴Cu-SARTATE in the SSTR2 over-expressing cell line A427-7 (closed symbols) and with an excess of Tyr³-octreotate (open symbols), for increasing periods of incubation.

FIG. 10: Comparison of the normalized uptake of ⁶⁴Cu-SARTATE in A427-7 and the A427 parental cell-line over 2 hours (p<0.0001).

FIG. 11: In vivo biodistribution of ⁶⁴Cu-SARTATE in select tissues from A427-7 tumour bearing Balb/c mice at 2 and 24 h. A blocking study was performed to confirm the specificity of ⁶⁴Cu-SARTATE for SSTR2 after 2 hours by co-injecting an excess of Tyr³-octreotate.

FIG. 12: In vivo PET imaging of ⁶⁴Cu-SARTATE using small animal PET maximum intensity projection images of A427-7 tumour-bearing Balb/c mice at 2 hours and 24 hours post-injection of ⁶⁴Cu-SARTATE, with and without the co-injection of an excess of Tyr³-octreotate.

### DETAILED DESCRIPTION

The present invention relates to stable formulations of a specific radioisotope-ligand complex. The present inventors have found that the formulations of a complex disclosed herein minimise dissociation of the radioisotope from the ligand and/or minimise radiolysis of the ligand arising from the radioisotope.

The formulations of a radioisotope-ligand complex referred to herein are stable in solution and under physiological conditions for a time. The stability of the formulation relates to the stability of the complex, where the radioisotope may undergo dissociation or the complex may undergo radiolysis. The stability of the complex can be measured by considering the radiochemical purity of the formulation. Radiochemical purity is defined as the amount of the radioisotope complexed by the sarcophagine ligand expressed as percentage of the total amount of the radioisotope present in the formulation. The radioisotope may be present in the formulation as a complex with the sarcophagine ligand, as a free radioisotope or as part of a radiolysis product.

It has previously been found that octreotate-containing ligands target somatostatin receptors, namely the type 2 (SSTR2) and type 5 (SSTR5) receptors. An example of a ligand containing octreotate is MeCOSar-octreotate, or MeCOSar-D-Phe-Cys-Tyr-D-Trp-Lys-Thr-Cys-Thr-OH, where MeCOSar is the macrocyclic sarcophagine ligand 5-[[8-amino-3,6,10,13,19-hexaazabicyclo-[6.6.6]eico-1-yl)amino]-5-oxo-pentanyl and octreotate is D-Phe-Cys-Tyr-D-Trp-Lys-Thr-Cys-Thr-OH. A person skilled in the art would appreciate that octreotate is a cyclic octapeptide and is derived from the corresponding linear peptide by formation of a Cys-Cys disulphide bond. A person skilled in the art would also appreciate that a sarcophagine (“sar”) is a nitrogen-containing hexadentate macrocyclic ligand, which is capable of complexing donor atoms, such as transition metal ions and in the context of the present invention Cu ions.

MeCOSar-octreotate (also referred to herein as “SARTATE”) is also as shown in Formula (I):

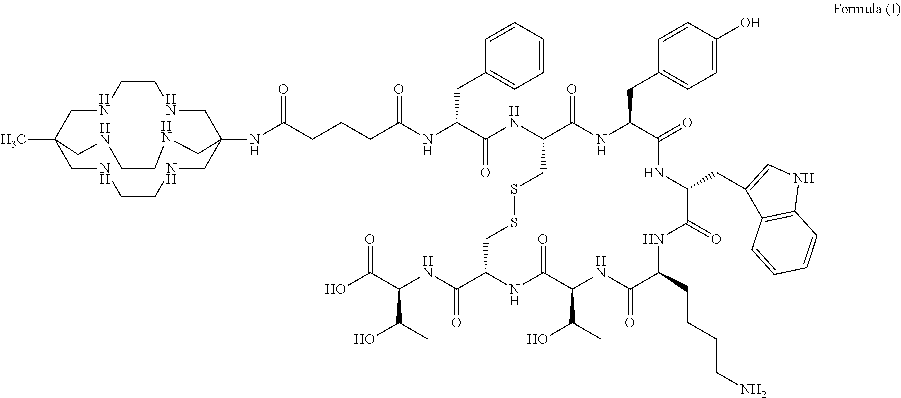 [SMILES: CC(O)[C@H](NC(=O)[C@@H]1CSSC[C@H](NC(=O)[C@@H](Cc2ccccc2)NC(=O)CCCC(=O)NC23CNCCNCC(C)(CNCCNC2)CNCCNC3)C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N1)C(=O)O]

The compound of Formula (I) may be produced via a coupling reaction between a sarcophagine ligand and the octreotate cyclic peptide, where the macrocyclic sarcophagine and the octreotate fragments are synthesised individually prior to coupling. The sarcophagine of Formula (I) is itself derived from an amino-capped macrocyclic ligand coupled with an aliphatic carboxylate group. The synthetic route to access the compound of Formula (I), and the component sarcophagine and octreotate fragments, has been previously disclosed in Dalton Trans., 2015, 43, 1386.

The present invention also contemplates the use of pharmaceutically acceptable salts of the compound of Formula (I), as part of the claimed formulations. Examples of pharmaceutically acceptable salts of compounds of Formula (I) may include the corresponding acetate salt, sodium salt, hydrochloride salt, potassium salt, magnesium salt, calcium salt or ammonium salt. In an embodiment, the compound of Formula (I) is provided as the acetate salt.

The administrable formulations of the present invention comprise a complex of a compound of Formula (I), or a salt thereof, and a radioisotope. The radioisotope, may also be referred to as a radionuclide, and may be a metal or a metal ion. The ligand of the present specification has been found to be particularly successful in complexing copper ions, especially Cu²⁺ ions. The complex of the Formula (I), comprising a copper ion radioisotope has been previously disclosed in Dalton Trans., 2015, 43, 1386. A person skilled in the art would also appreciate that a complex of Formula (I) and a radioisotope may be achieved by contacting the compound of Formula (I), or a salt thereof, with the radioisotope that is to be complexed, such that the compound of Formula (I), or a salt thereof, is complexed with the radioisotope. This may involve the mixing of the compound of Formula (I), or a salt thereof, and the radioisotope in a suitable solvent system (such as that specifically described herein).

In an embodiment, the ligand is complexed with a Cu ion. The copper ion may be radioactive, and thus a radionuclide or radioisotope of copper. In an embodiment, the ligand is complexed with ⁶⁰Cu. In another embodiment, the ligand is complexed with ⁶¹Cu. In another embodiment, the ligand is complexed with ⁶⁴Cu. In another embodiment, the ligand is complexed with ⁶⁷Cu. In a preferred embodiment, the ligand is complexed with ⁶⁴Cu. In another preferred embodiment, the ligand is complexed with ⁶⁷Cu.

The formulations of the present invention comprise ethanol as a component. The ethanol used in the formulation may be anhydrous ethanol. Alternatively, the ethanol used in the formulation may not have been subject to drying processes and may be hydrated. The ethanol is preferably pharmaceutical grade ethanol. The ethanol present in the formulation may further assist in preventing radiolysis of the radiolabelled complex of Formula (I).

In an embodiment, ethanol is present in the formulation in an amount of about 7% to about 13% (v/v). In an embodiment, ethanol is present in the formulation in an amount of about 7% (v/v). In another embodiment, ethanol is present in the formulation in an amount of about 8% (v/v). In another embodiment, ethanol is present in the formulation in an amount of about 9% (v/v). In another embodiment, ethanol is present in the formulation in an amount of about 10% (v/v). In another embodiment, ethanol is present in the formulation in an amount of about 11% (v/v). In another embodiment, ethanol is present in the formulation in an amount of about 12% (v/v). In another embodiment, ethanol is present in the formulation in an amount of about 13% (v/v). In a preferred embodiment, ethanol is present in the formulation in an amount of about 10% (v/v). In other embodiments, the present invention also contemplates ethanol in ranges between the aforementioned amounts.

The formulations of the present invention also comprise sodium chloride as a component. The sodium chloride in the formulations of the present invention may be provided as a saline solution. A saline solution is defined as an aqueous solution of sodium chloride. For example, normal saline is defined as an aqueous solution of sodium chloride at a concentration of 0.9% (w/v). In an embodiment of the present invention, the sodium chloride of a formulation is provided by a saline solution.

In an embodiment, sodium chloride is present in the formulation in an amount of about 0.6% to 1.2% (w/v). In an embodiment, sodium chloride is present in an amount of about 0.6% (w/v). In another embodiment, sodium chloride is present in an amount of about 0.7% (w/v). In another embodiment, sodium chloride is present in an amount of about 0.8% (w/v). In another embodiment, sodium chloride is present in an amount of about 0.9% (w/v). In another embodiment, sodium chloride is present in an amount of about 1.0% (w/v). In another embodiment, sodium chloride is present in an amount of about 1.1% (w/v). In another embodiment, sodium chloride is present in an amount of about 1.2% (w/v). In a preferred embodiment, sodium chloride is present in the formulation in an amount of about 0.9% (w/v). In other embodiments, the present invention also contemplates sodium chloride in ranges between the aforementioned amounts.

The formulations of the present invention comprise gentisic acid, or pharmaceutically acceptable salts and/or hydrates thereof, as a component. Gentisic acid is also known as 2,5-dihydroxybenzoic acid, 5-hydroxysalicylic acid or hydroquinonecarboxylic acid. Salts of gentisic acid may include the sodium salt and the sodium salt hydrate. Any reference to gentisic acid may include a reference to salts thereof, where relevant. It has been identified by the present inventors that the gentisic acid, or salt thereof, within the present formulations assists in preventing or minimising radiolysis of the radiolabelled complex of Formula (I).

In an embodiment, gentisic acid, or a salt thereof, is present in the formulation in an amount of about 0.02% to about 0.1% (w/v). In an embodiment, gentisic acid, or a salt thereof, is present in the formulation in an amount of about 0.02% (w/v). In another embodiment, gentisic acid, or a salt thereof, is present in the formulation in an amount of about 0.025% (w/v). In another embodiment, gentisic acid, or a salt thereof, is present in the formulation in an amount of about 0.03% (w/v). In another embodiment, gentisic acid, or a salt thereof, is present in the formulation in an amount of about 0.035% (w/v). In another embodiment, gentisic acid, or a salt thereof, is present in the formulation in an amount of about 0.04% (w/v). In another embodiment, gentisic acid, or a salt thereof, is present in the formulation in an amount of about 0.045% (w/v). In another embodiment, gentisic acid, or a salt thereof, is present in the formulation in an amount of about 0.05% (w/v). In another embodiment, gentisic acid, or a salt thereof, is present in the formulation in an amount of about 0.055% (w/v). In another embodiment, gentisic acid, or a salt thereof, is present in the formulation in an amount of about 0.6% (w/v). In another embodiment, gentisic acid, or a salt thereof, is present in the formulation in an amount of about 0.065% (w/v). In another embodiment, gentisic acid, or a salt thereof, is present in the formulation in an amount of about 0.07% (w/v). In another embodiment, gentisic acid, or a salt thereof, is present in the formulation in an amount of about 0.075% (w/v). In another embodiment, gentisic acid, or a salt thereof, is present in the formulation in an amount of about 0.08% (w/v).). In another embodiment, gentisic acid, or a salt thereof, is present in the formulation in an amount of about 0.085% (w/v). In another embodiment, gentisic acid, or a salt thereof, is present in the formulation in an amount of about 0.09% (w/v). In another embodiment, gentisic acid, or a salt thereof, is present in the formulation in an amount of about 0.095% (w/v). In another embodiment, gentisic acid, or a salt thereof, is present in the formulation in an amount of about 0.1% (w/v). In other embodiments, the present invention also contemplates gentisic acid, or a salt thereof, in ranges between the aforementioned amounts. In a preferred embodiment, gentisic acid, or a salt thereof, is present in the formulation in an amount of not more than 0.056% (w/v).

The formulations of the present invention have a pH of about 4 to about 8. A person skilled in the art would understand that the pH of the formulation is an inherent characteristic of the formulation, attributed to the combination of the compound of Formula (I) or a complex thereof, and the remaining excipients of the formulation. The present inventors have found that this pH range provides for optimal radiolabelling efficiency.

In an embodiment, the pH of the formulation is from about 4 to about 8. In an embodiment, the pH of the formulation is about 4. In another embodiment, the pH of the formulation is about 4.5. In another embodiment, the pH of the formulation is about 5.0. In an embodiment, the pH of the formulation is about 5.5. In another embodiment, the pH of the formulation is about 5.6. In another embodiment, the pH of the formulation is about 5.7. In another embodiment, the pH of the formulation is about 5.8. In another embodiment, the pH of the formulation is about 5.9. In another embodiment, the pH of the formulation is about 6.0. In another embodiment, the pH of the formulation is about 6.1. In another embodiment, the pH of the formulation is about 6.2. In another embodiment, the pH of the formulation is about 6.3. In another embodiment, the pH of the formulation is about 6.4. In another embodiment, the pH of the formulation is about 6.5. In another embodiment, the pH of the formulation is about 7.0. In another embodiment, the pH of the formulation is about 7.5. In another embodiment, the pH of the formulation is about 8.0. In a preferred embodiment, the pH of the formulation is about 6.0.

In a preferred embodiment, the aqueous formulation of the present invention comprises a compound of Formula (I), or a salt thereof, complexed with a Cu ion, about 10% (v/v) ethanol, about 0.9% (w/v) sodium chloride and about 0.06% gentisic acid, or a salt thereof, wherein the formulation has a pH of about 6.0. In an embodiment, the aqueous formulation of the present invention comprises a compound of Formula (I), or a salt thereof, complexed with a Cu ion, about 10% (v/v) ethanol, about 0.9% (w/v) sodium chloride, not more than 0.056% gentisic acid, or a salt thereof, wherein the formulation has a pH of about 6.0. In a further embodiment, the aqueous formulation of the present invention comprises a compound of Formula (I), or a salt thereof, complexed with a Cu ion, about 10% (v/v) ethanol, about 0.9% (w/v) sodium chloride and 0.056% gentisic acid, or a salt thereof, wherein the formulation has a pH of about 6.0. One skilled in the art would appreciate that the amount of the Formula (I)-Cu ion complex present in the aqueous formulation can be modified to suit varying needs.

In an embodiment, the aqueous formulation of the present invention comprises a compound of Formula (I), or a salt thereof, complexed with a ⁶⁴Cu ion, about 10% ethanol, about 0.9% (w/v) sodium chloride and about 0.06% gentisic acid, or a salt thereof, wherein the formulation has a pH of about 6.0. In an embodiment, the aqueous formulation of the present invention comprises a compound of Formula (I), or a salt thereof, complexed with a ⁶⁴Cu ion, about 10% ethanol, about 0.9% (w/v) sodium chloride and not more than 0.056% gentisic acid, or a salt thereof, wherein the formulation has a pH of about 6.0. In a further embodiment, the aqueous formulation of the present invention comprises a compound of Formula (I), or a salt thereof, complexed with a ⁶⁴Cu ion, about 10% (v/v) ethanol, about 0.9% (w/v) sodium chloride and 0.056% gentisic acid, or a salt thereof, wherein the formulation has a pH of about 6.0.

In an embodiment, the aqueous formulation of the present invention comprises a compound of Formula (I), or a salt thereof, complexed with a ⁶⁷Cu ion, about 10% ethanol, about 0.9% (w/v) sodium chloride and about 0.06% gentisic acid, or a salt thereof, wherein the formulation has a pH of about 6.0. In an embodiment, the aqueous formulation of the present invention comprises a compound of Formula (I), or a salt thereof, complexed with a ⁶⁷Cu ion, about 10% ethanol, about 0.9% (w/v) sodium chloride and not more than 0.056% gentisic acid, or a salt thereof, wherein the formulation has a pH of about 6.0. In a further embodiment, the aqueous formulation of the present invention comprises a compound of Formula (I), or a salt thereof, complexed with a ⁶⁷Cu ion, about 10% (v/v) ethanol, about 0.9% (w/v) sodium chloride and 0.056% gentisic acid, or a salt thereof, wherein the formulation has a pH of about 6.0.

In an embodiment, the aqueous formulation of the present invention comprises a compound of Formula (I) as the acetate salt, complexed with a ⁶⁴Cu ion, about 10% ethanol, about 0.9% (w/v) sodium chloride and about 0.06% gentisic acid, or a salt thereof, wherein the formulation has a pH of about 6.0. In another embodiment, the aqueous formulation of the present invention comprises a compound of Formula (I) as the acetate salt, complexed with a ⁶⁴Cu ion, about 10% ethanol, about 0.9% (w/v) sodium chloride and about 0.056% gentisic acid, or a salt thereof, wherein the formulation has a pH of about 6.0. In another embodiment, the aqueous formulation of the present invention comprises a compound of Formula (I) as the acetate, salt, complexed with a ⁶⁴Cu ion, about 10% ethanol, about 0.9% (w/v) sodium chloride and not more than 0.056% gentisic acid, or a salt thereof, wherein the formulation has a pH of about 6.0.

In an embodiment, the aqueous formulation of the present invention comprises a compound of Formula (I) as the acetate salt, complexed with a ⁶⁷Cu ion, about 10% ethanol, about 0.9% (w/v) sodium chloride and about 0.06% gentisic acid, or a salt thereof, wherein the formulation has a pH of about 6.0. In another embodiment, the aqueous formulation of the present invention comprises a compound of Formula (I) as the acetate salt, complexed with a ⁶⁷Cu ion, about 10% ethanol, about 0.9% (w/v) sodium chloride and about 0.056% gentisic acid, or a salt thereof, wherein the formulation has a pH of about 6.0. In another embodiment, the aqueous formulation of the present invention comprises a compound of Formula (I) as the acetate, salt, complexed with a ⁶⁷Cu ion, about 10% ethanol, about 0.9% (w/v) sodium chloride and not more than 0.056% gentisic acid, or a salt thereof, wherein the formulation has a pH of about 6.0.

The aqueous formulation of the present invention may also comprise an acetate salt as a buffering salt. The acetate salt may be ammonium acetate or sodium acetate.

The present inventors have also found that the formulation may be further stabilised with the addition of L-methionine, or a salt thereof. The addition of L-methionine to a formulation comprising a compound of Formula (I), ethanol, sodium chloride and gentisic acid or a salt thereof, further enhances the stability of the formulation by preventing or minimising radiolysis of a radiolabelled complex of Formula (I). The present inventors have also found that the addition of L-methionine to a formulation comprising a compound of Formula (I) and a Cu ion allows for a formulation with a higher starting radioactivity to be obtained, where the Cu ion is a radioisotope of Cu.

Accordingly, the present invention also provides an aqueous formulation for parenteral administration comprising a compound of Formula (I), or a salt thereof, complexed with a Cu ion:

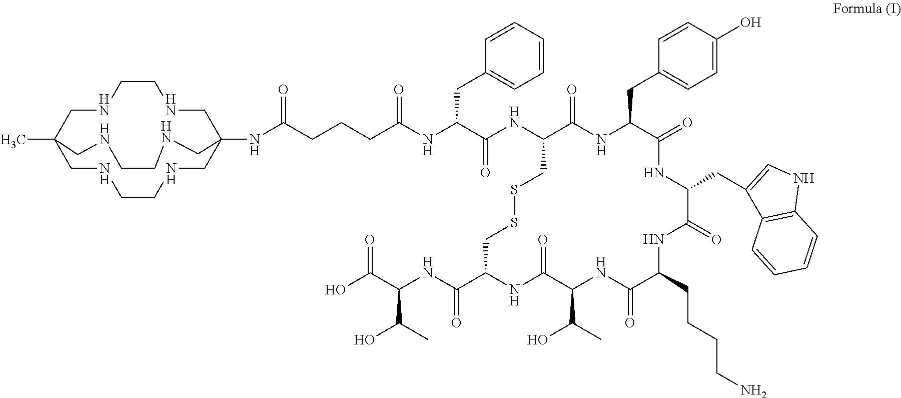 [SMILES: CC(O)[C@H](NC(=O)[C@@H]1CSSC[C@H](NC(=O)[C@@H](Cc2ccccc2)NC(=O)CCCC(=O)NC23CNCCNCC(C)(CNCCNC2)CNCCNC3)C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N1)C(=O)O]

the formulation further comprising:
about 7 to about 13% (v/v) ethanol;
about 0.3 to about 1.2% (w/v) sodium chloride;
about 0.02 to about 0.1% (w/v) gentisic acid or a salt thereof; and
about 1 to about 4 mg/mL L-methionine or a salt thereof;
   wherein the formulation has a pH of between about 4 to about 8.

In an embodiment, L-methionine, or a salt thereof, is present in the formulation in an amount of about 1 mg/mL to about 4 mg/mL. In an embodiment, L-methionine, or a salt thereof, is present in the formulation in an amount of about 1.0 mg/mL. In another embodiment, L-methionine, or a salt thereof, is present in the formulation in an amount of about 1.5 mg/mL. In another embodiment, L-methionine, or a salt thereof, is present in the formulation in an amount of about 2.0 mg/ml. In another embodiment, L-methionine, or a salt thereof, is present in the formulation in an amount of about 2.5 mg/mL. In another embodiment, L-methionine, or a salt thereof, is present in the formulation in an amount of about 3.0 mg/mL. In another embodiment, L-methionine, or a salt thereof, is present in the formulation in an amount of about 3.5 mg/ml. In another embodiment, L-methionine, or a salt thereof, is present in the formulation in an amount of about 4.0 mg/mL.

In a further embodiment, the aqueous formulation of the present invention comprises a compound of Formula (I), or a salt thereof, complexed with a Cu ion, about 10% (v/v) ethanol, about 0.9% (w/v) sodium chloride, about 0.06% gentisic acid, or a salt thereof, and about 2.5 mg/mL L-methionine, or a salt thereof, wherein the formulation has a pH of about 6.0. In another embodiment, the aqueous formulation of the present invention comprises a compound of Formula (I), or a salt thereof, complexed with a Cu ion, about 10% (v/v) ethanol, about 0.9% (w/v) sodium chloride, not more than 0.056% gentisic acid, or a salt thereof, and about 2.5 mg/mL L-methionine, or a salt thereof, wherein the formulation has a pH of about 6.0. In a further embodiment, the aqueous formulation of the present invention comprises a compound of Formula (I), or a salt thereof, complexed with a Cu ion, about 10% (v/v) ethanol, about 0.9% (w/v) sodium chloride, 0.056% gentisic acid, or a salt thereof, and about 2.5 mg/mL L-methionine, or a salt thereof, wherein the formulation has a pH of about 6.0. One skilled in the art would appreciate that the amount of the Formula (I)-Cu ion complex present in the aqueous formulation can be modified to suit varying needs.

In a further embodiment, the aqueous formulation of the present invention comprises a compound of Formula (I), or a salt thereof, complexed with a ⁶⁴Cu ion, about 10% (v/v) ethanol, about 0.9% (w/v) sodium chloride, about 0.06% gentisic acid, or a salt thereof, and about 2.5 mg/mL L-methionine, or a salt thereof, wherein the formulation has a pH of about 6.0. In an embodiment, the aqueous formulation of the present invention comprises a compound of Formula (I), or a salt thereof, complexed with a ⁶⁴Cu ion, about 10% (v/v) ethanol, not more than 0.9% (w/v) sodium chloride, not more than 0.056% gentisic acid, or a salt thereof, and about 2.5 mg/mL L-methionine, or a salt thereof, wherein the formulation has a pH of about 6.0. In a further embodiment, the aqueous formulation of the present invention comprises a compound of Formula (I), or a salt thereof, complexed with a ⁶⁴Cu ion, about 10% (v/v) ethanol, about 0.9% (w/v) sodium chloride, about 0.056% gentisic acid, or a salt thereof, and about 2.5 mg/mL L-methionine, or a salt thereof, wherein the formulation has a pH of about 6.0.

In a further embodiment, the aqueous formulation of the present invention comprises a compound of Formula (I), or a salt thereof, complexed with a ⁶⁷Cu ion, about 10% (v/v) ethanol, about 0.9% (w/v) sodium chloride, about 0.06% gentisic acid, or a salt thereof, and about 2.5 mg/mL L-methionine, or a salt thereof, wherein the formulation has a pH of about 6.0. In an embodiment, the aqueous formulation of the present invention comprises a compound of Formula (I), or a salt thereof, complexed with a ⁶⁷Cu ion, about 10% (v/v) ethanol, not more than 0.9% (w/v) sodium chloride, not more than 0.056% gentisic acid, or a salt thereof, and about 2.5 mg/mL L-methionine, or a salt thereof, wherein the formulation has a pH of about 6.0. In a further embodiment, the aqueous formulation of the present invention comprises a compound of Formula (I), or a salt thereof, complexed with a ⁶⁷Cu ion, about 10% (v/v) ethanol, about 0.9% (w/v) sodium chloride, about 0.056% gentisic acid, or a salt thereof, and about 2.5 mg/mL L-methionine, or a salt thereof, wherein the formulation has a pH of about 6.0. In a further embodiment, the aqueous formulation of the present invention comprises a compound of Formula (I), or a salt thereof, complexed with a ⁶⁷Cu ion, about 10% (v/v) ethanol, about 0.9% (w/v) sodium chloride, about 0.056% gentisic acid, or a salt thereof, and about 2.5 mg/mL L-methionine, or a salt thereof, wherein the formulation has a pH of about 6.0.

In a further embodiment, the aqueous formulation of the present invention comprises a compound of Formula (I) as the acetate salt, complexed with a ⁶⁴Cu ion, about 10% (v/v) ethanol, about 0.9% (w/v) sodium chloride, about 0.06% gentisic acid, or a salt thereof, and about 2.5 mg/mL L-methionine, or a salt thereof, wherein the formulation has a pH of about 6.0. In another embodiment, the aqueous formulation of the present invention comprises a compound of Formula (I) as the acetate salt, complexed with a ⁶⁴Cu ion, about 10% (v/v) ethanol, about 0.9% (w/v) sodium chloride, about 0.056% gentisic acid, or a salt thereof, and about 2.5 mg/mL L-methionine, or a salt thereof, wherein the formulation has a pH of about 6.0. In another embodiment, the aqueous formulation of the present invention comprises a compound of Formula (I) as the acetate, salt, complexed with a ⁶⁴Cu ion, about 10% ethanol, about 0.9% (w/v) sodium chloride, not more than 0.056% gentisic acid, or a salt thereof, and about 2.5 mg/mL L-methionine, or a salt thereof, wherein the formulation has a pH of about 6.0.

In a further embodiment, the aqueous formulation of the present invention comprises a compound of Formula (I) as the acetate salt, complexed with a ⁶⁷Cu ion, about 10% (v/v) ethanol, about 0.9% (w/v) sodium chloride, about 0.06% gentisic acid, or a salt thereof, and about 2.5 mg/mL L-methionine, or a salt thereof, wherein the formulation has a pH of about 6.0. In another embodiment, the aqueous formulation of the present invention comprises a compound of Formula (I) as the acetate salt, complexed with a ⁶⁷Cu ion, about 10% (v/v) ethanol, about 0.9% (w/v) sodium chloride, about 0.056% gentisic acid, or a salt thereof, and about 2.5 mg/mL L-methionine, or a salt thereof, wherein the formulation has a pH of about 6.0. In another embodiment, the aqueous formulation of the present invention comprises a compound of Formula (I) as the acetate, salt, complexed with a ⁶⁷Cu ion, about 10% ethanol, about 0.9% (w/v) sodium chloride, not more than 0.056% gentisic acid, or a salt thereof, and about 2.5 mg/mL L-methionine, or a salt thereof, wherein the formulation has a pH of about 6.0.

According to the present invention, a formulation of a complex of ⁶⁴Cu and a compound of Formula (I) may have a radiochemical purity of at least about 90% for a time of at least 45 hours. This means that at least about 90% of the ⁶⁴Cu radioisotope present in the formulation is complexed with the compound of Formula (I), or a salt thereof, for at least 45 hours after preparation of the formulation. Where the ⁶⁴Cu radioisotope present in the formulation is not complexed with the compound of Formula (I), or a salt thereof, the ⁶⁴Cu radioisotope may be present as a free ⁶⁴Cu ion, or as part of a radiolysis product.

In an embodiment, the radiochemical purity of a formulation of the present invention comprising a complex of ⁶⁴Cu and a compound of Formula (I), or a salt thereof is about 90% at a time of about 45 hours after preparation of the formulation. In another embodiment, the radiochemical purity of a formulation of the present invention comprising a complex of ⁶⁴Cu and a compound of Formula (I), or a salt thereof is about 91% at a time of about 45 hours after preparation of the formulation. In another embodiment, the radiochemical purity of a formulation of the present invention comprising a complex of ⁶⁴Cu and a compound of Formula (I), or a salt thereof is about 92% at a time of about 45 hours after preparation of the formulation.

In another embodiment, the radiochemical purity of a formulation of the present invention comprising a complex of ⁶⁴Cu and a compound of Formula (I), or a salt thereof is about 93% at a time of about 45 hours after preparation of the formulation. In another embodiment, the radiochemical purity of a formulation of the present invention comprising a complex of ⁶⁴Cu and a compound of Formula (I), or a salt thereof is about 94% at a time of about 45 hours after preparation of the formulation. In another embodiment, the radiochemical purity of a formulation of the present invention comprising a complex of ⁶⁴Cu and a compound of Formula (I), or a salt thereof is about 95% at a time of about 45 hours after preparation of the formulation. In another embodiment, the radiochemical purity of a formulation of the present invention comprising a complex of ⁶⁴Cu and a compound of Formula (I), or a salt thereof is about 96% at a time of about 45 hours after preparation of the formulation. In another embodiment, the radiochemical purity of a formulation of the present invention comprising a complex of ⁶⁴Cu and a compound of Formula (I), or a salt thereof is about 97% at a time of about 45 hours after preparation of the formulation. In another embodiment, the radiochemical purity of a formulation of the present invention comprising a complex of ⁶⁴Cu and a compound of Formula (I), or a salt thereof is about 98% at a time of about 45 hours after preparation of the formulation. In another embodiment, the radiochemical purity of a formulation of the present invention comprising a complex of ⁶⁴Cu and a compound of Formula (I), or a salt thereof is about 99% at a time of about 45 hours after preparation of the formulation.

In an embodiment, the radiochemical purity of a formulation of the present invention comprising a complex of ⁶⁴Cu and a compound of Formula (I), or a salt thereof is about 99% immediately after preparation of the formulation. In another embodiment, the radiochemical purity of a formulation of the present invention comprising a complex of ⁶⁴Cu and a compound of Formula (I), or a salt thereof is about 99% after about 1 h after preparation of the formulation. In another embodiment, the radiochemical purity of a formulation of the present invention comprising a complex of ⁶⁴Cu and a compound of Formula (I), or a salt thereof is about 99% after about 3 h after preparation of the formulation. In another embodiment, the radiochemical purity of a formulation of the present invention comprising a complex of ⁶⁴Cu and a compound of Formula (I), or a salt thereof is about 99% after about 6 h after preparation of the formulation. In another embodiment, the radiochemical purity of a formulation of the present invention comprising a complex of ⁶⁴Cu and a compound of Formula (I), or a salt thereof is about 99% after about 9 h after preparation of the formulation. In another embodiment, the radiochemical purity of a formulation of the present invention comprising a complex of ⁶⁴Cu and a compound of Formula (I), or a salt thereof is about 99% after about 12 h after preparation of the formulation. In another embodiment, the radiochemical purity of a formulation of the present invention comprising a complex of ⁶⁴Cu and a compound of Formula (I), or a salt thereof is about 99% after about 15 h after preparation of the formulation. In another embodiment, the radiochemical purity of a formulation of the present invention comprising a complex of ⁶⁴Cu and a compound of Formula (I), or a salt thereof is about 99% after about 18 h after preparation of the formulation. In another embodiment, the radiochemical purity of a formulation of the present invention comprising a complex of ⁶⁴Cu and a compound of Formula (I), or a salt thereof is about 99% after about 21 h after preparation of the formulation. In another embodiment, the radiochemical purity of a formulation of the present invention comprising a complex of ⁶⁴Cu and a compound of Formula (I), or a salt thereof is about 99% after about 24 h after preparation of the formulation.

According to the present invention, a formulation of a complex of ⁶⁷Cu and a compound of Formula (I) may also have a radiochemical purity of at least 90% for a time of at least 11 hours. This means that at least about 90% of the ⁶⁷Cu radioisotope present in the formulation is complexed with the compound of Formula (I), or a salt thereof, for at least 11 hours after preparation of the formulation. Where the ⁶⁷Cu radioisotope present in the formulation is not complexed with the compound of Formula (I), or a salt thereof, the ⁶⁷Cu radioisotope may be present as a free ⁶⁷Cu ion, or as part of a radiolysis product.

In an embodiment, the radiochemical purity of a formulation of the present invention comprising a complex of ⁶⁷Cu and a compound of Formula (I), or a salt thereof is about 90% at a time of about 11 hours after preparation of the formulation. In another embodiment, the radiochemical purity of a formulation of the present invention comprising a complex of ⁶⁷Cu and a compound of Formula (I), or a salt thereof is about 91% at a time of about 11 hours after preparation of the formulation. In another embodiment, the radiochemical purity of a formulation of the present invention comprising a complex of ⁶⁷Cu and a compound of Formula (I), or a salt thereof is about 92% at a time of about 11 hours after preparation of the formulation. In another embodiment, the radiochemical purity of a formulation of the present invention comprising a complex of ⁶⁷Cu and a compound of Formula (I), or a salt thereof is about 93% at a time of about 11 hours after preparation of the formulation. In another embodiment, the radiochemical purity of a formulation of the present invention comprising a complex of ⁶⁷Cu and a compound of Formula (I), or a salt thereof is about 94% at a time of about 11 hours after preparation of the formulation. In another embodiment, the radiochemical purity of a formulation of the present invention comprising a complex of ⁶⁷Cu and a compound of Formula (I), or a salt thereof is about 95% at a time of about 11 hours after preparation of the formulation. In another embodiment, the radiochemical purity of a formulation of the present invention comprising a complex of ⁶⁷Cu and a compound of Formula (I), or a salt thereof is about 96% at a time of about 11 hours after preparation of the formulation. In another embodiment, the radiochemical purity of a formulation of the present invention comprising a complex of ⁶⁷Cu and a compound of Formula (I), or a salt thereof is about 97% at a time of about 11 hours after preparation of the formulation. In another embodiment, the radiochemical purity of a formulation of the present invention comprising a complex of ⁶⁷Cu and a compound of Formula (I), or a salt thereof is about 98% at a time of about 11 hours after preparation of the formulation. In another embodiment, the radiochemical purity of a formulation of the present invention comprising a complex of ⁶⁷Cu and a compound of Formula (I), or a salt thereof is about 99% at a time of about 11 hours after preparation of the formulation.

In an embodiment, the radiochemical purity of a formulation of the present invention comprising a complex of ⁶⁷Cu and a compound of Formula (I), or a salt thereof is about 99% immediately after preparation of the formulation. In another embodiment, the radiochemical purity of a formulation of the present invention comprising a complex of ⁶⁷Cu and a compound of Formula (I), or a salt thereof is about 99% after about 1 h after preparation of the formulation. In another embodiment, the radiochemical purity of a formulation of the present invention comprising a complex of ⁶⁷Cu and a compound of Formula (I), or a salt thereof is about 99% after about 3 h after preparation of the formulation. In another embodiment, the radiochemical purity of a formulation of the present invention comprising a complex of ⁶⁷Cu and a compound of Formula (I), or a salt thereof is about 99% after about 6 h after preparation of the formulation. In another embodiment, the radiochemical purity of a formulation of the present invention comprising a complex of ⁶⁷Cu and a compound of Formula (I), or a salt thereof is about 99% after about 9 h after preparation of the formulation. In another embodiment, the radiochemical purity of a formulation of the present invention comprising a complex of ⁶⁷Cu and a compound of Formula (I), or a salt thereof is about 99% after about 12 h after preparation of the formulation. In another embodiment, the radiochemical purity of a formulation of the present invention comprising a complex of ⁶⁷Cu and a compound of Formula (I), or a salt thereof is about 99% after about 15 h after preparation of the formulation. In another embodiment, the radiochemical purity of a formulation of the present invention comprising a complex of ⁶⁷Cu and a compound of Formula (I), or a salt thereof is about 99% after about 18 h after preparation of the formulation. In another embodiment, the radiochemical purity of a formulation of the present invention comprising a complex of ⁶⁷Cu and a compound of Formula (I), or a salt thereof is about 99% after about 21 h after preparation of the formulation. In another embodiment, the radiochemical purity of a formulation of the present invention comprising a complex of ⁶⁷Cu and a compound of Formula (I), or a salt thereof is about 99% after about 24 h after preparation of the formulation.

### Preparation of an Aqueous Formulation of the Present Invention

The compound of Formula (I), or a salt thereof, complexed with a Cu ion may be provided by mixing a compound of Formula (I), or a salt thereof, with a solution of a Cu ion in the presence of a buffering solution. The solution may then be filtered and subsequently washed to provide the formulation comprising a compound of Formula (I), or a salt thereof, complexed with a Cu ion. Accordingly, the present invention provides a process for preparing an aqueous formulation comprising a compound of Formula (I) complexed with a Cu ion, the method comprising the steps of:
i) preparing a buffering solution of an acetate salt, wherein the buffering solution further comprises ethanol and gentisic acid or a salt thereof;
ii) dissolving a compound of Formula (I), or a salt thereof, in the buffering solution obtained from step i);
iii) adding a solution of a Cu ion to the solution obtained from step ii);
iv) filtering the solution obtained from step iii) on to a stationary phase; and
v) washing the stationary phase of step iv) with ethanol and saline;
   to recover an aqueous formulation comprising a compound of Formula (I), or a salt thereof, complexed with a Cu ion.

The buffering solution may be a solution of ammonium acetate. Alternatively, the buffering solution may be a solution of sodium acetate. A buffering solution employing an acetate salt is used to maintain the pH in a range that allows for maximum and rapid complexation of a compound of Formula (I), or a salt thereof, with a Cu ion. The buffering solution may comprise an aqueous solution of ammonium acetate at a concentration of between about 0.08 to about 0.12 mol/L. In an embodiment, the buffering solution comprises an aqueous solution of ammonium acetate at a concentration of about 0.08 mol/L. In another embodiment, the buffering solution comprises an aqueous solution of ammonium acetate at a concentration of about 0.09 mol/L. In another embodiment, the buffering solution comprises an aqueous solution of ammonium acetate at a concentration of about 0.1 mol/L. In another embodiment, the buffering solution comprises an aqueous solution of ammonium acetate at a concentration of about 0.11 mol/L. In another embodiment, the buffering solution comprises an aqueous solution of ammonium acetate at a concentration of about 0.12 mol/L. In a preferred embodiment, the buffering solution comprises an aqueous solution of 0.1 mol/L.

The buffering solution also comprises ethanol as a component. As previously described, the ethanol may be anhydrous or may be previously subjected to drying procedures known in the art. The buffering solution may comprise ethanol at a concentration of between about 3 to about 11% (v/v). In an embodiment, the buffering solution comprises ethanol at a concentration of about 3% (v/v). In another embodiment, the buffering solution comprises ethanol at a concentration of about 3.5% (v/v). In another embodiment, the buffering solution comprises ethanol at a concentration of about 4% (v/v). In another embodiment, the buffering solution comprises ethanol at a concentration of about 4.5% (v/v). In another embodiment, the buffering solution comprises ethanol at a concentration of about 5% (v/v). In another embodiment, the buffering solution comprises ethanol at a concentration of about 6% (v/v). In another embodiment, the buffering solution comprises ethanol at a concentration of about 7% (v/v). In another embodiment, the buffering solution comprises ethanol at a concentration of about 8% (v/v). In another embodiment, the buffering solution comprises ethanol at a concentration of about 9% (v/v). In another embodiment, the buffering solution comprises ethanol at a concentration of about 10% (v/v). In another embodiment, the buffering solution comprises ethanol at a concentration of about 10% (v/v). In another embodiment, the buffering solution comprises ethanol at a concentration of about 11% (v/v). In a preferred embodiment, the buffering solution comprises ethanol at a concentration of about 10% (v/v).

The buffering solution also comprises gentisic acid, or a salt thereof, as a component. As previously described, salts of gentisic acid may include the sodium salt or the sodium salt hydrate. Other salts of gentisic acid are also contemplated. The buffering solution may comprise sodium gentisate at a concentration of between about 0.1 to about 0.55% (w/v). In an embodiment, the buffering solution comprises sodium gentisate at a concentration of about 0.1% (w/v). In another embodiment, the buffering solution comprises sodium gentisate at a concentration of about 0.15% (w/v). In another embodiment, the buffering solution comprises sodium gentisate at a concentration of about 0.2% (w/v). In another embodiment, the buffering solution comprises sodium gentisate at a concentration of about 0.25% (w/v). In another embodiment, the buffering solution comprises sodium gentisate at a concentration of about 0.3% (w/v). In another embodiment, the buffering solution comprises sodium gentisate at a concentration of about 0.35% (w/v). In another embodiment, the buffering solution comprises sodium gentisate at a concentration of about 0.4% (w/v). In another embodiment, the buffering solution comprises sodium gentisate at a concentration of about 0.45% (w/v). In another embodiment, the buffering solution comprises sodium gentisate at a concentration of about 0.5% (w/v). In another embodiment, the buffering solution comprises sodium gentisate at a concentration of about 0.55% (w/v). In a preferred embodiment, the buffering solution comprises sodium gentisate at a concentration of about 0.228% (w/v).

According to an embodiment of the present invention, the buffering solution may be prepared by mixing ethanol and gentisic acid, or a salt thereof, with an aqueous solution of ammonium acetate. The buffering solution may be prepared by sequentially adding ethanol and gentisic acid, or a salt thereof, to the aqueous solution of ammonium acetate, or alternatively, the ethanol and gentisic acid, or a salt thereof, may be added to the solution of ammonium acetate together. In an embodiment of the present invention, the buffering solution comprises ammonium acetate at a concentration of about 0.1 M, with ethanol at a concentration of about 4-11% (v/v) and gentisic acid, or a salt thereof, at a concentration of about 0.5% (w/v).

According to an embodiment of the present invention, a compound of Formula (I), or a salt thereof, is mixed with a buffering solution of aqueous ammonium acetate comprising ethanol and gentisic acid, or a salt thereof. The compound of Formula (I) or a salt thereof, may be obtained as a solid. In an embodiment, the compound of Formula (I) or a salt thereof, is obtained as a lyophilised powder. In an embodiment, the compound of Formula (I) or a salt thereof, obtained as a lyophilised powder is mixed with a buffering solution of aqueous ammonium acetate comprising ethanol and gentisic acid or a salt thereof. In an embodiment, about 15 μg to about 65 μg of the compound of Formula (I) or a salt thereof, as a lyophilised powder is mixed with a buffering solution of aqueous ammonium acetate comprising ethanol and gentisic acid or a salt thereof. In another embodiment, about 15 μg of the compound of Formula (I) or a salt thereof, as a lyophilised powder is mixed with a buffering solution of aqueous ammonium acetate comprising ethanol and gentisic acid or a salt thereof. In another embodiment, about 20 μg of the compound of Formula (I) or a salt thereof, as a lyophilised powder is mixed with a buffering solution of aqueous ammonium acetate comprising ethanol and gentisic acid, or a salt thereof. In another embodiment, about 25 μg of the compound of Formula (I), or a salt thereof, as a lyophilised powder is mixed with a buffering solution of aqueous ammonium acetate comprising ethanol and gentisic acid, or a salt thereof. In another embodiment, about 30 μg of the compound of Formula (I), or a salt thereof, as a lyophilised powder is mixed with a buffering solution of aqueous ammonium acetate comprising ethanol and gentisic acid, or a salt thereof. In another embodiment, about 35 μg of the compound of Formula (I), or a salt thereof, as a lyophilised powder is mixed with a buffering solution of aqueous ammonium acetate comprising ethanol and gentisic acid, or a salt thereof. In another embodiment, about 40 μg of the compound of Formula (I), or a salt thereof, as a lyophilised powder is mixed with a buffering solution of aqueous ammonium acetate comprising ethanol and gentisic acid, or a salt thereof. In another embodiment, about 45 μg of the compound of Formula (I), or a salt thereof, as a lyophilised powder is mixed with a buffering solution of aqueous ammonium acetate comprising ethanol and gentisic acid, or a salt thereof. In another embodiment, about 50 μg of the compound of Formula (I), or a salt thereof, as a lyophilised powder is mixed with a buffering solution of aqueous ammonium acetate comprising ethanol and gentisic acid, or a salt thereof. In another embodiment, about 55 μg of the compound of Formula (I), or a salt thereof, as a lyophilised powder is mixed with a buffering solution of aqueous ammonium acetate comprising ethanol and gentisic acid, or a salt thereof. In another embodiment, about 60 μg of the compound of Formula (I), or a salt thereof, as a lyophilised powder is mixed with a buffering solution of aqueous ammonium acetate comprising ethanol and gentisic acid, or a salt thereof. In another embodiment, about 65 μg of the compound of Formula (I), or a salt thereof, as a lyophilised powder is mixed with a buffering solution of aqueous ammonium acetate comprising ethanol and gentisic acid, or a salt thereof.

A solution of a Cu ion is added to the mixture of a compound of Formula (I), or a salt thereof, and the buffering solution of aqueous ammonium acetate comprising ethanol and gentisic acid, or a salt thereof, and is allowed to stand for a time.

In an embodiment, the solution of a Cu ion is a solution of a Cu salt. In another embodiment, the solution of a Cu ion is a solution of a chloride salt containing copper. In another embodiment, the solution of a Cu ion is a solution of a copper(II) chloride salt. In another embodiment, the solution of a Cu ion is a solution of a copper salt containing a ⁶⁰Cu radioisotope. In another embodiment, the solution of a Cu ion is a solution of a chloride salt containing a ⁶¹Cu radioisotope. In another embodiment, the solution of a Cu ion is a solution of a chloride salt containing a ⁶⁴Cu radioisotope. In another embodiment, the solution of a Cu ion is a solution of a chloride salt containing a ⁶⁷Cu radioisotope. In another embodiment, the solution of a Cu ion is a solution of a radioactive copper(II) chloride salt. In another embodiment, the solution of a Cu ion is a solution of a copper(II) chloride salt, wherein the copper is the ⁶¹Cu isotope. In another embodiment, the solution of a Cu ion is a solution of a copper(II) chloride salt, wherein the copper is the ⁶⁴Cu isotope. In another embodiment, the solution of a Cu ion is a solution of a copper(II) chloride salt, wherein the copper is the ⁶⁷Cu isotope. In another embodiment, the solution of Cu ion is a solution of [⁶¹Cu]CuCl₂. In another embodiment, the solution of a Cu ion is a solution of [⁶⁴Cu]CuCl₂. In another embodiment, the solution of Cu ion is a solution of [⁶⁷Cu]CuCl₂.

The solution of a Cu ion is provided as an aqueous solution. The Cu ion may be provided in an aqueous solution of hydrochloric acid. In an embodiment, the Cu ion is provided in a solution of between about 0.01 to about 0.1 mol/L hydrochloric acid. In an embodiment, the Cu ion is provided in a solution of about 0.01 mol/L hydrochloric acid. In another embodiment, the Cu ion is provided in a solution of about 0.02 mol/L hydrochloric acid. In another embodiment, the Cu ion is provided in a solution of about 0.05 mol/L hydrochloric acid. In another embodiment, the Cu ion is provided in a solution of about 0.075 mol/L hydrochloric acid. In another embodiment, the Cu ion is provided in a solution of about 0.1 mol/L hydrochloric acid. In a preferred embodiment, the Cu ion is provided as [⁶⁴Cu]CuCl₂ in a solution of about 0.05 mol/L hydrochloric acid. In another preferred embodiment, the Cu ion is provided as [⁶⁷Cu]CuCl₂ in a solution of about 0.05 mol/L hydrochloric acid.

The solution of a ⁶⁴Cu-radioisotope is provided as an aqueous solution with a radioactivity of between about 750 to about 3500 MBq. In an embodiment, the radioactivity of the ⁶⁴Cu-radioisotope solution is about 750 MBq. In another embodiment, the radioactivity of the ⁶⁴Cu-radioisotope solution is about 1000 MBq. In another embodiment, the radioactivity of the ⁶⁴Cu-radioisotope solution is about 1250 MBq. In another embodiment, the radioactivity of the ⁶⁴Cu-radioisotope solution is about 1500 MBq. In another embodiment, the radioactivity of the ⁶⁴Cu-radioisotope solution is about 1750 MBq. In another embodiment, the radioactivity of the ⁶⁴Cu-radioisotope solution is about 2000 MBq. In another embodiment, the radioactivity of the ⁶⁴Cu-radioisotope solution is about 2250 MBq. In another embodiment, the radioactivity of the ⁶⁴Cu-radioisotope solution is about 2500 MBq. In another embodiment, the radioactivity of the ⁶⁴Cu-radioisotope solution is about 2750 MBq. In another embodiment, the radioactivity of the ⁶⁴Cu-radioisotope solution is about 3000 MBq. In another embodiment, the radioactivity of the ⁶⁴Cu-radioisotope solution is about 3250 MBq. In another embodiment, the radioactivity of the ⁶⁴Cu-radioisotope is about 3500 MBq.

The solution of a 67Cu-radioisotope is provided as an aqueous solution with a radioactivity of between about 1000 to about 5000 MBq. In an embodiment, the radioactivity of the ⁶⁷Cu-radioisotope is about 1000 MBq. In another embodiment, the radioactivity of the ⁶⁷Cu-radioisotope is about 1500 MBq. In another embodiment, the radioactivity of the ⁶⁷Cu-radioisotope is about 2000 MBq. In another embodiment, the radioactivity of the ⁶⁷Cu-radioisotope is about 2500 MBq. In another embodiment, the radioactivity of the ⁶⁷Cu-radioisotope is about 3000 MBq. In another embodiment, the radioactivity of the ⁶⁷Cu-radioisotope is about 3500 MBq. In another embodiment, the radioactivity of the ⁶⁷Cu-radioisotope is about 4000 MBq. In another embodiment, the radioactivity of the ⁶⁷Cu-radioisotope is about 4500 MBq. In another embodiment, the radioactivity of the ⁶⁷Cu-radioisotope is about 5000 MBq.

A mixture of a Cu ion, a compound of Formula (I), or a salt thereof, and the buffering solution of aqueous ammonium acetate comprising ethanol and gentisic acid, or a salt thereof, may be allowed to stand at room temperature. The mixture may be allowed to stand with stirring, alternatively, the mixture is allowed to stand without stirring. The mixture may be allowed to stand for a time between about 5 to about 25 minutes. In an embodiment, the mixture of a Cu ion, a compound of Formula (I), or a salt thereof, and the buffering solution of aqueous ammonium acetate comprising ethanol and gentisic acid is allowed to stand without stirring for about 5 minutes. In another embodiment, the mixture of a Cu ion, a compound of Formula (I), or a salt thereof, and the buffering solution of aqueous ammonium acetate comprising ethanol and gentisic acid is allowed to stand without stirring for about 10 minutes. In another embodiment, the mixture of a Cu ion, a compound of Formula (I), or a salt thereof, and the buffering solution of aqueous ammonium acetate comprising ethanol and gentisic acid is allowed to stand without stirring for about 15 minutes. In another embodiment, the mixture of a Cu ion, a compound of Formula (I), or a salt thereof, and the buffering solution of aqueous ammonium acetate comprising ethanol and gentisic acid is allowed to stand without stirring for about 20 minutes. In another embodiment, the mixture of a Cu ion, a compound of Formula (I), or a salt thereof, and the buffering solution of aqueous ammonium acetate comprising ethanol and gentisic acid is allowed to stand without stirring for about 25 minutes. In a preferred embodiment, the mixture of a Cu ion, a compound of Formula (I), or a salt thereof, and the buffering solution of aqueous ammonium acetate comprising ethanol and gentisic acid is allowed to stand without stirring for about 15 minutes. In another preferred embodiment, the mixture of a ⁶⁴Cu-radioisotope, a compound of Formula (I), or a salt thereof, and the buffering solution of aqueous ammonium acetate comprising ethanol and gentisic acid is allowed to stand without stirring for about 20 minutes.

According to another embodiment of the present invention, the mixture of a Cu ion, a compound of Formula (I), or a salt thereof, and the buffering solution of aqueous ammonium acetate comprising ethanol and gentisic acid, or a salt thereof, is filtered. The mixture may be filtered to remove the acetate salt that may remain in the solution. The mixture may be filtered through a solid phase extraction process. The mixture may be filtered through a solid phase extraction process, where the stationary phase of the solid phase extraction cartridge retains the compound of Formula (I), or a salt thereof, complexed with a Cu ion, any compound of Formula (I), or a salt thereof, that is not complexed and some gentisic acid in the form of a salt that is present, such as sodium gentisate. As used herein, the term “stationary phase” refers to a resin-like material that is held within the solid phase extraction cartridge and allows for the separation of compounds based on their polarity.

The solid phase extraction process as described herein may use a reverse-phase stationary phase. As used herein, the term “reverse-phase” in relation to a stationary phase refers to a stationary phase that is hydrophobic in nature, such that the stationary phase has an affinity for hydrophobic or uncharged molecules. Examples of a reverse-phase stationary phase may include Phenomenex Strata-X 33u Polymeric Reversed Phase, Waters tC18 or Waters C18. Other similar stationary phases may be used. As the solid phase extraction process uses a reverse-phase stationary phase, the ammonium acetate from the buffering solution, any free Cu ions and the majority of the remaining gentisic acid or its salt is not retained by the stationary phase and these components are discarded.

In an embodiment, the mixture of a Cu ion, a compound of Formula (I) and the buffering solution of aqueous ammonium acetate is filtered through a solid phase extraction cartridge. In an embodiment, the mixture of a Cu ion, a compound of Formula (I) and the buffering solution of aqueous ammonium acetate, is filtered through a solid phase extraction cartridge with a reverse-phase stationary phase. In an embodiment, the ammonium acetate and gentisic acid from the buffering solution is removed by a solid phase extraction cartridge with a reverse-phase stationary phase. In an embodiment, the compound of Formula (I) complexed with a Cu ion is retained by a solid phase extraction cartridge with a reverse-phase stationary phase. In a preferred embodiment, the mixture of a ⁶⁴Cu-radioisotope, a compound of Formula (I) and the buffering solution of aqueous ammonium acetate is filtered through a solid phase extraction cartridge with a reverse-phase stationary phase. In a preferred embodiment, the compound of Formula (I) complexed with a ⁶⁴Cu ion is retained by a solid phase extraction cartridge with a reverse-phase stationary phase. In another preferred embodiment, the mixture of a ⁶⁷Cu-radioisotope, a compound of Formula (I) and the buffering solution of aqueous ammonium acetate is filtered through a solid phase extraction cartridge with reverse-phase stationary phase. In another preferred embodiment, the compound of Formula (I) complexed with a ⁶⁷Cu ion is retained by a solid phase extraction cartridge with a reverse-phase stationary phase.

The compound of Formula (I) complexed with a Cu ion is eluted from the solid phase extraction cartridge containing the stationary phase by washing with a solvent. As the solid phase extraction cartridge contains a reverse-phase stationary phase, eluting the compound of Formula (I) complexed with a Cu ion requires washing of the stationary phase with ethanol, saline and/or another solvent. In an embodiment, the solid phase extraction cartridge is washed with ethanol to elute the compound of Formula (I) complexed with a Cu ion. In another embodiment, the solid phase extraction cartridge is washed with saline to elute the compound of Formula (I) complexed with a Cu ion. In another embodiment, the solid phase extraction cartridge is washed with ethanol and saline to elute the compound of Formula (I) complexed with a Cu ion. In a preferred embodiment, the solid phase extraction cartridge is washed with ethanol and saline sequentially to elute the compound of Formula (I) complexed with a Cu ion. In a preferred embodiment, the solid phase extraction cartridge is washed with ethanol and saline sequentially to provide the formulation of the present invention. In a preferred embodiment, the solid phase extraction cartridge is washed with ethanol and saline sequentially to elute the compound of Formula (I) complexed with a Cu ion and any retained components, such as gentisic acid or its salt.

As discussed above, the present inventors have found that formulations of Formula (I) complexed with a Cu ion further comprising L-methionine show even greater stability towards radiolysis. In another preferred embodiment, the solid phase extraction cartridge is washed with ethanol and saline sequentially to elute the compound of Formula (I) complexed with a Cu ion and gentisic acid, or a salt thereof, into a solution of L-methionine in saline. In another preferred embodiment, the solid phase extraction cartridge is washed with ethanol and saline sequentially to elute the compound of Formula (I) complexed with a Cu ion, ammonium acetate and gentisic acid, or a salt thereof, into a solution of L-methionine in saline. In another preferred embodiment, the concentration of L-methionine in the saline solution into which the solid phase extraction cartridge is washed is about 2.5 mg/ml. In another preferred embodiment, the solid phase extraction cartridge is washed with ethanol and saline sequentially to provide a formulation of the present invention.

A person skilled in the art would understand that the excipients of the formulation include the solvent that is used to elute the compound of Formula (I) complexed with a Cu ion from the stationary phase, and that the amount of each solvent used is related to the amount of each excipient in the formulations of the present invention.

A person skilled in the art would understand that the present disclosure provides a manual process for producing a formulation according to the present invention. A person skilled in the art would understand that the steps described herein may be automated, by using a suitable automated radiosynthesis module, in order to obtain a formulation according to the present invention.

The present inventors have found that the formulations disclosed herein have greater stability and show reduced radiolysis in light of the higher starting radioactivity. This enhanced stability may be attributed to the increased radiochemical purity of the formulation at a given radioactivity. The stability of the formulations of the present invention may be observed for a time of up to 45 hours post-manufacture for a formulation of ⁶⁴Cu-SARTATE and up to 11 hours post-manufacture for a formulation of ⁶⁷Cu-SARTATE. Where the formulations of the present invention are used for the purposes of treatment or therapy, the greater stability may mean that doses for multiple patients at multiple remote locations can be prepared at the same time at a single facility. This may mean that resources for manufacture are required at a single facility, rather than at multiple facilities, and greater efficiency in production of the formulations may be achieved. Where the formulations of the present invention are used for imaging purposes, further advantages may be provided since the clinical imaging sites can receive a dosage form that is ready to inject. This may be particularly advantageous for clinical sites where dedicated radiopharmaceutical production facilities do not exist.

The formulations of the present invention comprise a ligand-radioisotope complex, where the ligand is a compound of Formula (I), or a salt thereof. The compound of Formula (I), or a salt thereof, and the radioisotope may be supplied in separate containers. Alternatively, the compound of Formula (I), or a salt thereof, and the radioisotope may be supplied together as a ligand-radioisotope complex.

The container consisting of the compound of Formula (I), or a salt thereof, may provide the compound of Formula (I), or a salt thereof, as a lyophilised powder. The container may be provided at a temperature of between −20° C. and 20° C.

The formulations may be provided as a kit comprising a container of the radioisotope and a separate container with the ligand and instructions for making the aqueous formulation of the present invention. In an embodiment, the kit of the present invention comprises a container providing a solution of a ⁶⁴Cu radioisotope and a separate container providing a compound of Formula (I), or a salt thereof. The container providing the radioisotope may contain a solution of a metal salt where the metal is a radionuclide.

In an embodiment, a kit of the present invention comprises a container with a solution of a ⁶⁴Cu radioisotope. In a further embodiment, a kit of the present invention comprises a container with a solution of a copper salt containing a ⁶⁴Cu radioisotope. In another embodiment, a kit of the present invention comprises a container with a solution of a chloride salt containing a ⁶⁴Cu radioisotope. In another embodiment, a kit of the present invention comprises a container with a solution of a radioactive copper(II) chloride salt. In another embodiment, a kit of the present invention comprises a container with a solution of a copper(II) chloride salt, wherein the copper ion is the ⁶⁴Cu isotope. In another embodiment, a kit of the present invention comprises a container with a solution of [⁶⁴Cu]CuCl₂.

In an embodiment, a kit of the present invention comprises a container with a solution of ⁶⁷Cu radioisotope. In another embodiment, a kit of the present invention comprises a container with a solution of a copper salt containing a ⁶⁷Cu radioisotope. In another embodiment, a kit of the present invention comprises a container with a solution of a chloride salt containing a ⁶⁷Cu radioisotope. In another embodiment, a kit of the present invention comprises a container with a solution of a radioactive copper(II) chloride salt. In another embodiment, a kit of the present invention comprises a container with a solution of a copper(II) chloride salt, wherein the copper ion is the ⁶⁷Cu isotope. In another embodiment, a kit of the present invention comprises a container with a solution of [⁶⁷Cu]CuCl₂.

The solution of the radioisotope is typically provided as an aqueous solution. In an embodiment, a kit of the present invention provides a radioisotope in the form of an aqueous solution. In a further embodiment, a kit of the present invention provides a radioisotope in the form of an acidic aqueous solution. In another embodiment, a kit of the present invention provides a radioisotope as a solution in hydrochloric acid. The radioisotope may be provided as a solution in hydrochloric acid at a concentration of between about 0.01 and about 0.1 mol/L.

In an embodiment, a kit of the present invention comprises a container with a solution of [⁶⁴Cu]CuCl₂ in hydrochloric acid. In an embodiment, a kit of the present invention comprises a container with a solution of [⁶⁴Cu]CuCl₂ in hydrochloric acid, wherein the hydrochloric acid is at a concentration of about 0.02 mol/L. In an embodiment, a kit of the present invention comprises a container with a solution of [⁶⁴Cu]CuCl₂ in hydrochloric acid, wherein the hydrochloric acid is at a concentration of about 0.05 mol/L. In an embodiment, a kit of the present invention comprises a container with a solution of [⁶⁴Cu]CuCl₂ in hydrochloric acid, wherein the hydrochloric acid is at a concentration of about 0.1 mol/L.

In an embodiment, a kit of the present invention comprises a container with a solution of [⁶⁷Cu]CuCl₂ in hydrochloric acid. In another embodiment, a kit of the present invention comprises a container with a solution of [⁶⁷Cu]CuCl₂ in hydrochloric acid, wherein the hydrochloric acid is at a concentration of about 0.02 mol/L. In another embodiment, a kit of the present invention comprises a container with a solution of [⁶⁷Cu]CuCl₂ in hydrochloric acid, wherein the hydrochloric acid is at a concentration of about 0.05 mol/L. In another embodiment, a kit of the present invention comprises a container with a solution of [⁶⁷Cu]CuCl₂ in hydrochloric acid, wherein the hydrochloric acid is at a concentration of about 0.1 mol/L.

The kit may further comprise a container consisting of ethanol, sodium chloride and gentisic acid in a buffered solution. This container may provide ethanol, sodium chloride and gentisic acid in an aqueous solution, or alternatively, the container may consist only of ethanol, sodium chloride and gentisic acid. In an embodiment, the kit comprises a container consisting of ethanol, sodium chloride and gentisic acid, or a salt thereof, in an ammonium acetate buffering solution.

The kit may also comprise a container consisting of ethanol, sodium chloride, gentisic acid, or a salt thereof, and L-methionine, or a salt thereof, in a buffered solution. The container of the kit may provide ethanol, sodium chloride, gentisic acid or a salt thereof, and L-methionine or a salt thereof in an aqueous solution, or alternatively, the container may consist only of ethanol, sodium chloride, gentisic acid or a salt thereof and L-methionine or a salt thereof. In an embodiment, the kit comprises a container consisting of ethanol, sodium chloride, gentisic acid, or a salt thereof, and L-methionine, or a salt thereof. In an embodiment, the kit comprises a container consisting of ethanol, sodium chloride, gentisic acid, or a salt thereof, and L-methionine, or a salt thereof, in an ammonium acetate buffering solution.

### Uses of a Formulation of the Present Invention

Formulations of the present invention may be particularly useful for the purposes of diagnosis and treatment in medicine. Complexes with a ligand bearing an appropriate targeting fragment can be used to locate specific tissue types. For such complexes to be considered suitable for use in in vivo diagnosis and treatment, the complex must display appropriate kinetic, stability and clearance properties under physiological conditions, in addition to the requisite solubility and stability properties of the complex in solution. As used herein, the term “complex” may relate to a ligand-metal ion complex, where the metal ion is a radioactive isotope or alternatively, the metal ion is a non-radioactive isotope.

Accordingly, the present invention provides a method for radioimaging, a method for diagnosing a disease in a subject or a method for therapy of a disease in a subject, comprising administering to the subject an effective amount of a formulation as defined herein. The present inventors have found that the formulations of the present invention may be used in a method for radioimaging, a method for diagnosing or a method for therapy of a cancer.

As used herein the term “cancer” broadly encompasses a class of neoplastic diseases characterised with abnormal cell growth with the potential to invade or spread to other parts of the body. These are to be contrasted with benign tumours, which do not spread to other parts of the body and therefore the definition as used herein includes all malignant (cancerous) disease states. The term therefore encompasses the treatment of tumours.

Accordingly, the term “tumour” is used generally to define any malignant cancerous or pre-cancerous cell growth, and may include leukemias, but is particularly directed to solid tumours or carcinomas such as melanomas, colon, lung, ovarian, skin, breast, pancreas, pharynx, brain, prostate, CNS, and renal cancers (as well as other cancers).

Somatostatin receptors, especially SSTR2, are also highly expressed at the plasma membrane of certain tumours and cancers, including pancreatic, gastrointestinal and pulmonary neuroendocrine tumours (NETs), pituitary adenomas, breast carcinomas, meningiomas, neuroblastomas, medulloblastomas, phaeochromocytomas and paragangliomas. The presence of somatostatin receptors on such tumours has led to the development and clinical application of stable somatostatin receptors, e.g. compounds bearing an octreotate motif. The present inventors have found that a complex of a compound of Formula (I) and a Cu ion, as found in the formulations of the present invention, has shown particular utility in binding to somatostatin receptors and in particular, somatostatin receptors of subtype 2 and subtype 5. In certain embodiments, the formulation may be used in the radioimaging, the diagnosis or the treatment of a cancer where the somatostatin receptor is expressed or highly expressed.

The formulations of the present invention comprise a compound of Formula (I) containing an octreotate motif, which is analogous to octreotide, a clinically useful analogue of somatostatin. Somatostatin is released by neuroendocrine cells of the gastrointestinal tract and acts through 5 somatostatin receptor subtypes (SSTR1 to 5). Given the analogous nature of the octreotate motif to octreotide, the compounds of formula (I) may localise at and bind to particular sites where somatostatin receptors are present. Similarly, a compound of Formula (I) complexed with a Cu ion may also localise and bind to the same sites.

The radioisotope-ligand complex of the present invention may comprise a radioisotope such as ⁶⁴Cu. The ⁶⁴Cu isotope has a half-life of approximately 12.7 hours and decays by both positron emission and beta decay, which makes the use of a ⁶⁴Cu-labelled complex suitable for use in various modes of radioimaging. In particular, the decay characteristics and half-life of ⁶⁴Cu make this radioisotope a favourable choice for use in positron emission tomography (PET) and single-photon emission computed tomography (SPECT). The radioisotope-ligand complex of the present invention may comprise a radioisotope such as ⁶¹Cu. The ⁶¹Cu isotope has a half-life of approximately 3 hours and decays by positron emission, which makes the use of a ⁶¹Cu-labelled complex suitable for use in various modes of radioimaging. The radioisotope-ligand complex of the present invention may also comprise a radioisotope such as ⁶⁷Cu. The ⁶⁷Cu isotope has a half-life of approximately 61.8 hours and decays by beta emission, which makes the use of a ⁶⁷Cu-labelled complex suitable for use in SPECT imaging. The ⁶⁷Cu-labelled complex may also be suitable for use as a radiotherapy treatment.

The administration of an effective amount of a formulation comprising a compound of Formula (I) and a Cu radioisotope, such as ⁶⁰Cu, ⁶¹Cu, ⁶⁴Cu or ⁶⁷Cu, may lead to the binding of the complex of the compound of Formula (I) and the Cu radioisotope to somatostatin receptors. Where the somatostatin receptors are expressed on the surface of a tumour, the complex of a compound of Formula (I) and a Cu ion may bind to the somatostatin receptors. In an embodiment, the present invention provides a method for radioimaging, comprising administering to the subject a formulation comprising a compound of Formula (I) and a Cu ion. In an embodiment, a formulation comprising a compound of Formula (I) and a ⁶⁴Cu or ⁶⁷Cu ion may be used in a method for radioimaging. Monitoring of a subject to which a formulation comprising a compound of Formula (I) and a Cu radioisotope was administered by PET or SPECT, for example, allows for the visualisation and subsequent detection of tumour sites. The visualisation information obtained by radioimaging may provide information in relation to the location of any such tumour sites. Monitoring of the subject to which the radiolabelled complex was administered by SPECT, for example, allows for the visualisation and subsequent detection of tumour sites. This provides information in relation to the location of the tumours, where present. Repeated imaging at later timepoints allows for monitoring clearance of the radioisotope-ligand complex, which enables dosimetry estimates to be calculated. A person skilled in the art would understand that the amount to be administered in order to facilitate radioimaging may vary and will subsequently depend on the nature of the subject and the intended site of imaging.

In order for the complex to be suitable for radioimaging purposes, the radioisotope-ligand complex must display sufficient metabolic stability, i.e. that the complex remains intact with the radioisotope bound to the ligand, for a requisite time. The present invention provides a complex of a compound of Formula (I) and ⁶⁴Cu that remains intact for up to 45 hours, as evidenced by the absence of radioisotope loss and metabolic decomposition.

The formulations of the present invention may be administered to a subject for the purposes of radioimaging, diagnosis or therapy. Administration is by a parenteral route, with administration by intravenous injection preferred. Alternatively, the formulations of the present invention may be given by intraarterial or other routes, for delivery into the systemic circulation. The subject to which the formulation is administered is then placed into a PET scanner and images showing the localisation of the radioisotope-ligand complex, and subsequently location of any tumours, are obtained. This then allows for diagnosis and detection of tumours. Alternatively, a sample (for example, a blood or a tissue sample) that has been exposed to a formulation of the present invention may be analysed by gamma spectroscopy, gamma counting, liquid scintillation counting, autoradiography or beta probe in order to obtain radioimages.

In an embodiment, the present invention provides the use of a formulation comprising a compound of Formula (I) in a method for the radioimaging of a tumour or cancer. One skilled in the art would understand that the information obtained from radioimaging of a subject may be used in the diagnosis of a tumour or cancer in the subject. In an embodiment, the present invention provides a method for the diagnosis of a tumour or cancer. In a further embodiment, the tumour or cancer may be a somatostatin-receptor expressing tumour or cancer. In an embodiment, the tumour or cancer is a neuroendocrine tumour. In another embodiment, the tumour or cancer is a pituitary adenoma. In another embodiment, tumour or cancer is a neuroblastoma. In another embodiment, the tumour or cancer is a meningioma. In another embodiment, the tumour or cancer is a medulloblastoma. In another embodiment, the tumour or cancer is a breast carcinoma. In another embodiment, the tumour or cancer is a phaeochromocytoma. In another embodiment, the tumour or cancer is a paraganglioma. In another embodiment, the tumour is a pancreatic tumour. In another embodiment, the tumour is a gastrointestinal tumour.

Where the formulation of the present invention comprises a compound of Formula (I) and a Cu radioisotope, the administration of the formulation may treat a tumour or cancer. As discussed above, the compound of Formula (I) may bind somatostatin receptors on the surface of a tumour or cancer site, such the binding of the compound to locations with somatostatin receptors also brings the Cu radioisotope into close proximity of this location. As the Cu radioisotope undergoes radioactive decay, with the mode of decay dependent on the exact radioisotope chosen, the products of decay may be useful in the treatment of a tumour or cancer due to the proximity of the tumour or cancer to the compound of Formula (I) and Cu radioisotope.

In an embodiment, the present invention provides the use of a formulation comprising a compound of Formula (I) and a Cu radioisotope in a method for treatment of a tumour or cancer. In an embodiment, the tumour or cancer is a neuroendocrine tumour. In another embodiment, the tumour or cancer is a pituitary adenoma. In another embodiment, tumour or cancer is a neuroblastoma. In another embodiment, the tumour or cancer is a meningioma. In another embodiment, the tumour or cancer is a medulloblastoma. In another embodiment, the tumour or cancer is a breast carcinoma. In another embodiment, the tumour or cancer is a phaeochromocytoma. In another embodiment, the tumour or cancer is a paraganglioma. In another embodiment, the tumour is a pancreatic tumour. In another embodiment, the tumour is a gastrointestinal tumour.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### EXAMPLES

### Example 1—Preparation of a Low-Dose Formulation, ⁶⁴Cu-SARTATE Incorporating Ethanol and Sodium Gentisate as Excipients to Reduce Radiolysis

A buffer solution of 0.1 M ammonium acetate is prepared, where the buffer solution also contains ethanol at a concentration of 4-10% (v/v). The buffer solution also contains sodium gentisate, where a 5 mL volume of the buffer solution contains 38 mg of sodium gentisate.

The compound of Formula (I) is obtained as a lyophilised powder. 20 μg of the compound of Formula (I) in its lyophilised form is dissolved in 5 ml of the prepared buffer solution.

A solution of [⁶⁴Cu]CuCl₂ in 0.05 M hydrochloric acid is prepared, where a 300 UL volume of this solution contains 1500 MBq of [⁶⁴Cu]. A 300 μL volume of this [⁶⁴Cu]CuCl₂ solution is added to the solution containing the compound of Formula (I) and sodium gentisate in ammonium acetate buffer. This combined solution is allowed to stand, without stirring, at room temperature for 15 minutes.

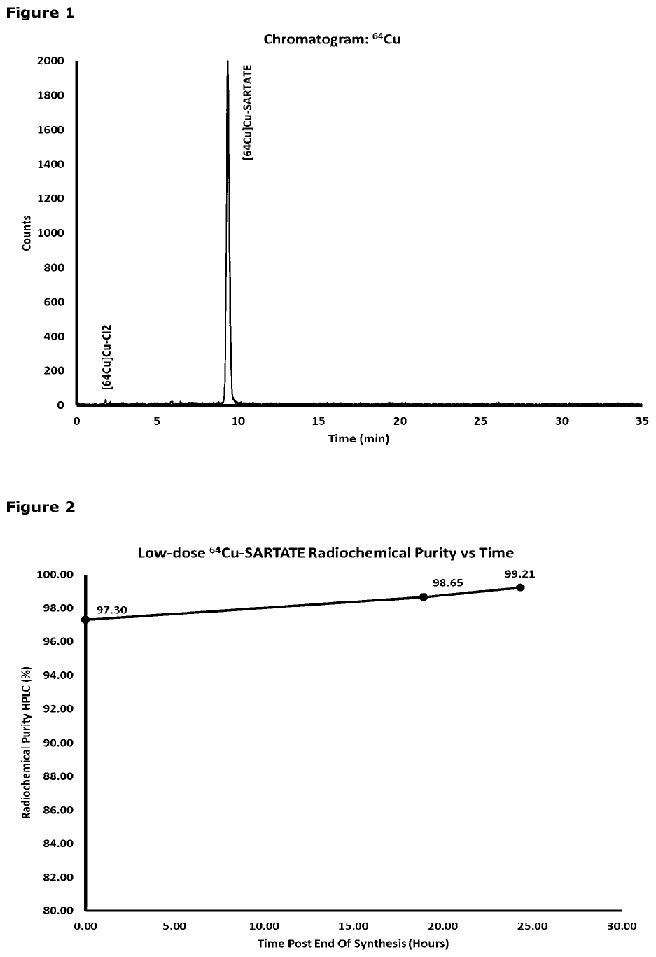

The solution is then filtered through a solid phase extraction cartridge. The cartridge is then eluted with 1.0 mL ethanol and then 9.0 mL saline solution into a sterile product vial, to give ⁶⁴Cu-SARTATE in a volume of 10 mL ethanol/saline solution. HPLC analysis of the solution obtained can be seen in FIG. 1, showing over 97% radiochemical purity. Further HPLC analysis of the same product solution obtained over multiple time points can be seen in FIG. 2, showing that the radiochemical purity remains >90% for more than 11 hours.

### Example 2—Preparation of a High-Dose ⁶⁴Cu-SARTATE Formulation, Incorporating Ethanol, Sodium Gentisate and L-Methionine as Excipients to Reduce Radiolysis

A buffer solution of 0.1 M ammonium acetate is prepared, where the buffer solution also contains ethanol at a concentration of 4-10% (v/v). The buffer solution also contains sodium gentisate, where a 5 mL volume of the buffer solution contains 114 mg of sodium gentisate.

The compound of Formula (I) is obtained as a lyophilised powder. 20 μg of the compound of Formula (I) in its lyophilised form is dissolved in 5 mL of the prepared buffer solution.

A solution of [⁶⁴Cu]CuCl₂ in 0.05 M hydrochloric acid is prepared, where a 300 μL volume of this solution contains 4650 MBq of [⁶⁴Cu]. A 300 μL volume of this [⁶⁴Cu]CuCl₂ solution is added to the solution containing the compound of Formula (I) and sodium gentisate in ammonium acetate buffer. This combined solution is allowed to stand, without stirring, at room temperature for 15 minutes.

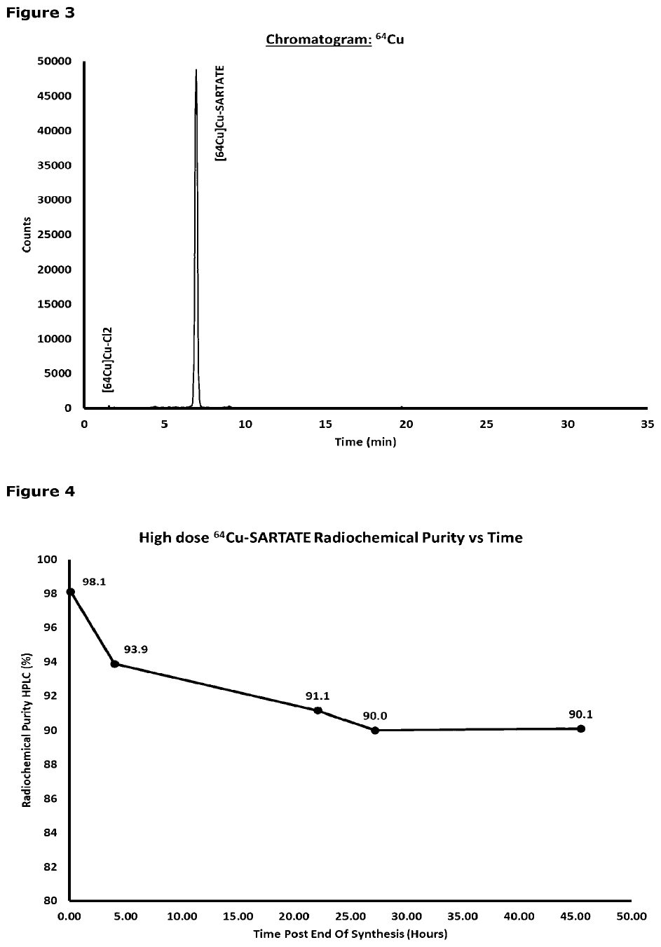

The solution is then filtered through a solid phase extraction cartridge. The cartridge is then eluted with 1.0 mL ethanol and then 16.0 mL saline solution, to give ⁶⁴Cu-SARTATE in a volume of 20 mL ethanol/saline solution. HPLC analysis of the solution obtained can be seen in FIG. 3, showing over 98% radiochemical purity. Further HPLC analysis of the same product solution obtained over multiple time points can be seen in FIG. 4, showing that the radiochemical purity remains >90% for more than 45 hours.

### Example 3—Preparation of a 67Cu-SARTATE Formulation, Incorporating Ethanol, Sodium Gentisate and L-Methionine as Excipients to Reduce Radiolysis

A buffer solution of 0.1 M ammonium acetate is prepared, where the buffer solution also contains ethanol at a concentration of 4-10% (v/v). The buffer solution also contains sodium gentisate, where a 5 mL volume of the buffer solution contains 114 mg of sodium gentisate.

The compound of Formula (I) is obtained as a lyophilised powder. 60 μg of the compound of Formula (I) in its lyophilised form is dissolved in 5 ml of the prepared buffer solution.

A solution of [⁶⁷Cu]CuCl₂ in 0.05 M hydrochloric acid is prepared, where a 300 μL volume of this solution contains 4650 MBq of [⁶⁴Cu]. A 300 μL volume of this [⁶⁷Cu]CuCl₂ solution is added to the solution containing the compound of Formula (I) and sodium gentisate in ammonium acetate buffer. This combined solution is allowed to stand, without stirring, at room temperature for 15 minutes.

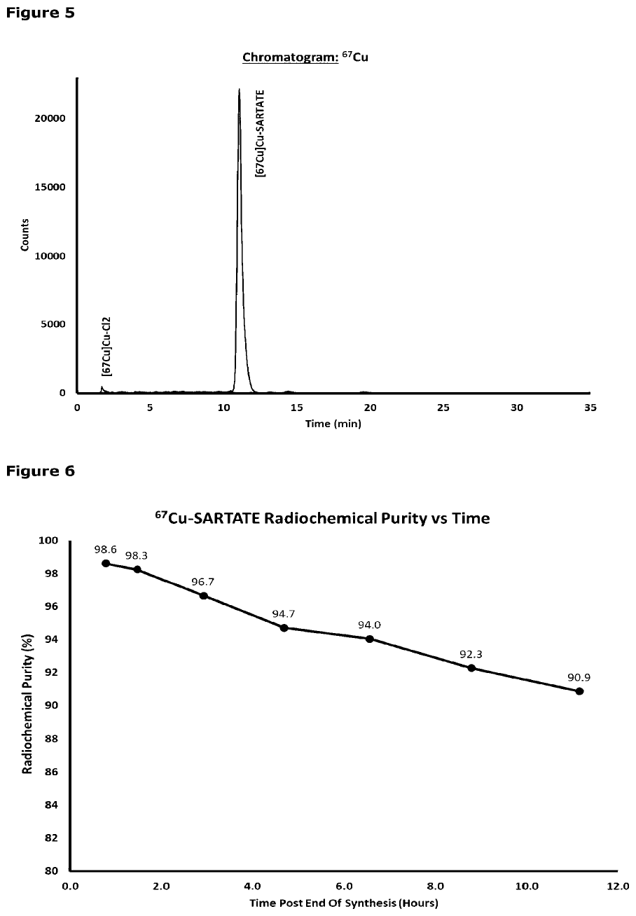

The solution is then filtered through a solid phase extraction cartridge. The cartridge is then eluted with 1.0 mL ethanol and then 16.0 mL saline solution into a sterile product vial containing a solution of L-methionine (50 mg in 3 mL saline solution), to give 67Cu-SARTATE in a volume of 20 mL ethanol/saline solution. HPLC analysis of the solution obtained can be seen in FIG. 5, showing over 98% radiochemical purity. Further HPLC analysis of the same product solution obtained over multiple time points can be seen in FIG. 6, showing that the radiochemical purity remains >90% for more than 11 hours.

### Example 4—In Vitro Serum Stability of ⁶⁴Cu-SARTATE

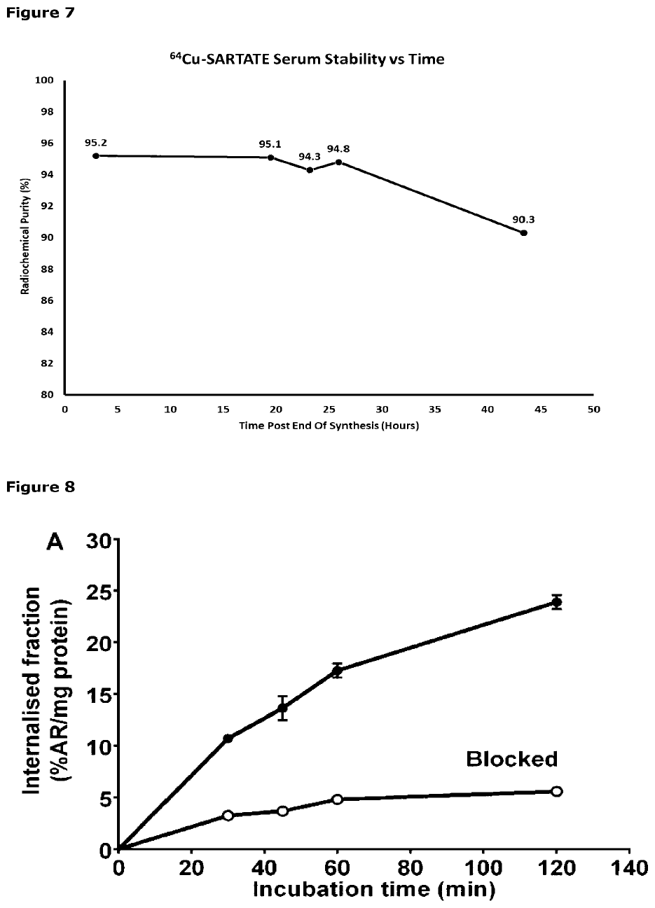

Incubation of ⁶⁴Cu-SARTATE (radiochemical purity >99%) with fresh human serum demonstrated high metabolic stability. HPLC analysis of the serum incubated with ⁶⁴Cu-SARTATE obtained can be seen in FIG. 7, indicating that >90% radioactivity in the non-protein bound fraction at 3 hrs, 20 hrs, 23 hrs, 26 hrs and 34 hrs was still chelator-bound representing intact radiopeptide and indicating no loss of copper or appreciable metabolic decomposition was detected for up to 43 hours.

### Example 5—In Vitro Internalisation and Cell-Surface Binding of ⁶⁴Cu-SARTATE

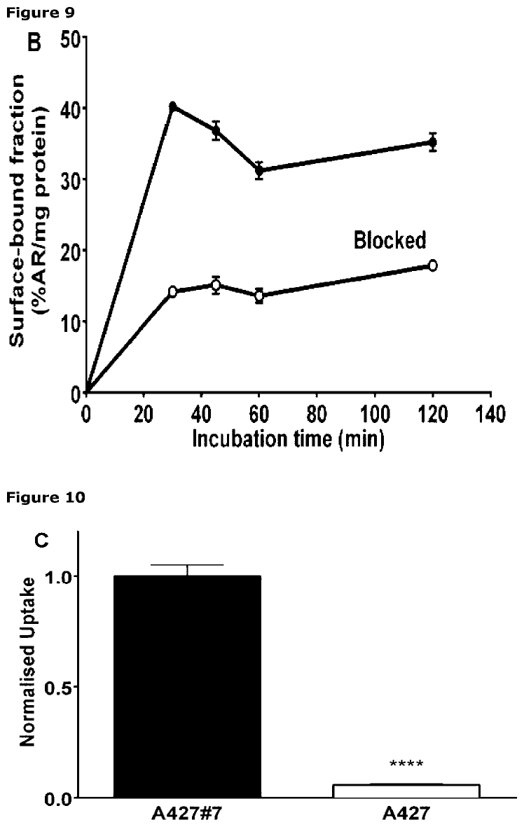

⁶⁴Cu-SARTATE internalisation and cell-surface binding studies were performed using A427-7 cells bearing somatostatin receptor 2. The percentage of total added radioactivity per mg of protein (% AR/mg protein) that was internalized increased with time, reaching 23.9±0.7 at 120 min (FIG. 8). Within 30 min, 40.2±0.7% AR/mg protein is bound to the cell surface (FIG. 9). This value decreased to 31.2±1.2 at 60 min and 35.2±1.3 at 120 min. Both receptor-mediated internalization and cell-surface binding was partially inhibited by the addition of cold Tyr³-octreotate to the medium. Normalized uptake of ⁶⁴Cu-SARTATE in the parental A427 cells was notably less than in the SSTR2 expressing A427-7 cells demonstrating the significance of receptor-specific accumulation (FIG. 10).

### Example 6—In Vivo Biodistribution of ⁶⁴Cu-SARTATE

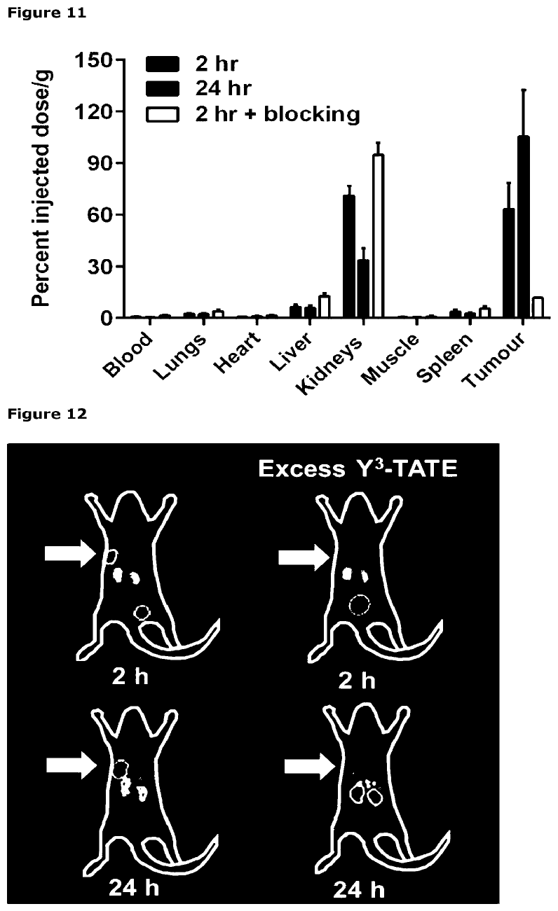

The biodistribution of Cu-SARTATE was investigated using ⁶⁴Cu-SARTATE in A427-7 tumour-bearing Balb/c nude mice (FIG. 11). ⁶⁴Cu-SARTATE had effective blood clearance at 2 hours (0.4±0.2% ID/g, where % ID/g is the percentage of the injected dose per gram of tissue) with further clearance at 24 hours (0.1±0.02% ID/g). Uptake of ⁶⁴Cu-SARTATE by the liver (3.1±1.3% ID/g) and kidneys (35.2±5.4% ID/g) was highest at 2 hours after dosing. By 24 hours after dosing, kidney uptake of ⁶⁴Cu SARTATE had fallen by 71% to 10.1±3.5% ID/g, suggesting effective renal clearance of ⁶⁴Cu-SARTATE. At 24 hours after dosing, uptake of ⁶⁴Cu-SARTATE in lungs and spleen (i.e., non-target organs) was 0.6±0.3% ID/g and 0.8±0.2% ID/g, respectively, while muscle accumulation was 0.1±0.01% ID/g at 24 hours. Tumour uptake of ⁶⁴Cu-SARTATE at 2 hours after administration was high at 31.2±13.1% ID/g and remained high at 24 hours to 31.4±14.0% ID/g. Co-administration of excess Tyr³-octreotate (XS Y³-TATE) to block the receptors significantly reduced tumour uptake of ⁶⁴Cu-SARTATE at 2 hours by 81% to 5.9±0.3% ID/g while increasing the non-target tissue uptake, as shown by a 135% increase in the kidneys to 47.7±6.3% ID/g.

### Example 7—In Vivo PET Imaging of ⁶⁴Cu-SARTATE

Small animal PET images of A427-7 tumour-bearing Balb/c mice at 2 and 24 hours, with and without blocking with an excess of Tyr³-octreotate are presented in FIG. 12. The tumour is clearly visible at 2 hours post-injection of ⁶⁴Cu-SARTATE with an average tumour to background ratio of 48. The tumour to background ratio at 24 hours remained constant at 45, which indicates a high degree of specific binding and stability of the complex. The co-administration of an excess of Tyr³-octreotate effectively blocked the tumour uptake, with tumour to background ratio of 3.1 at 2 hours and to below the limit of quantitation at 24 h. The blocking experiment further suggests the specificity for SSTR2 and the low level of non-specific binding of ⁶⁴Cu-SARTATE. Substantial uptake in the kidneys and bladder was evident in all animals suggesting renal clearance was the major excretion route. The tumour to kidneys ratio at 2 hours was 1.6 and increased to 2.8 at 24 hours.

### Example 8—In Vivo Toxicology of SARTATE

A single dose preclinical toxicology study in Sprague Dawley rats was conducted to evaluate the potential toxicity of SARTATE when administered via intravenous injection. Testing was performed on solutions of SARTATE-copper-complex (SCC) and unlabeled SARTATE ligand (SL) at a 1:1 ratio. The study was conducted according to the requirements of OECD GLP Principles.

The test item was administered once to six groups of 10 rats (5/sex) at three doses of 50, 250 and 1000 μg/kg in the vehicle at a volume of 3 mL/kg. Two vehicle control groups of 10 rats (5/sex) were administered the vehicle only (10% ethanol in 0.9% sodium chloride and 0.056% gentisic acid) at the same volumetric dose.

Four groups of rats (one vehicle and three test item treated 50, 250 and 1000 μg/kg) from the main study were sacrificed on Day 2. The remaining four groups of 10 rats (one vehicle and three test item treated 50, 250 and 1000 μg/kg) from the recovery study were observed for a treatment-free period of 14 days and sacrificed on Day 15 to assess reversibility of any toxicity.

The following parameters were evaluated: mortality, daily clinical observations, weekly body weights, weekly food consumption, haematology, biochemistry, urinalysis, organ weights and gross necropsy on day of sacrifice. Extensive histopathology was performed on all animals.

No mortalities related to treatment were observed in either the vehicle or the treated groups during both treatment and recovery periods. The test item produced no clinical abnormalities related to treatment in any animal during the 2-day and 15-day experimental periods. Treated and vehicle control groups displayed comparable body weights gains over the 2-day and 15-day experimental periods. Feed intake was similar in control and treated groups for the 2-day and 15-day experimental periods. Haematology, blood biochemistry and urine analysis revealed no test item-related effects. No macroscopic abnormalities were identified during the necropsy of all animals. There was no evidence of any test item-related effect on organ weight and all the tissues examined histopathologically in this study.

Under the conditions of the study, the test item administered intravenously at 50, 250 and 1000 μg/kg in the Sprague Dawley rat produced no toxic effects. The No Observed Adverse Effect level (NOAEL) is therefore 1000 μg/kg (1 mg/kg).

The NOAEL of 1 mg/kg in rats corresponds to a Human Equivalent Dose (HED) of 0.16 mg/kg, or a total dose of 11.2 mg in a patient with a weight of 70 kg. The maximum possible total dose in this clinical trial will be 0.02 mg (20 micrograms) per patient. The NOAEL therefore represents a safety margin of 50 times the maximum human dose of SARTATE. As the dose of ⁶⁴Cu-SARTATE to be administered to patients is determined by activity (200 MBq), it is expected that the likely dose of SARTATE actually injected will be a fraction of the total possible dose, which increases the safety margin substantially.

### Example 9—In Vitro Genotoxicity of SARTATE

To evaluate the mutagenic potential of SARTATE, GLP AMES testing was performed on solutions of SARTATE-copper-complex (SCC) and unlabeled SARTATE ligand (SL) at a 1:1 ratio. The SL:SCC solution did not induce an appropriate fold increase in the mean revertants per plate over the mean revertants per plate of the appropriate vehicle control. SL:SCC solution did not exhibit any cytotoxicity at the dose levels used with any of the 5 tester strains. The product is considered to be non-mutagenic.

1. An aqueous formulation for parenteral administration comprising a compound of Formula (I), or a salt thereof, complexed with a Cu ion 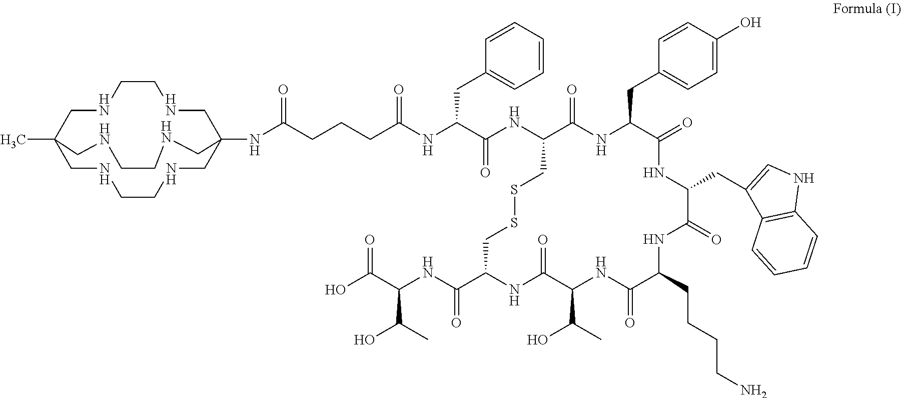 [SMILES: CC(O)[C@H](NC(=O)[C@@H]1CSSC[C@H](NC(=O)[C@@H](Cc2ccccc2)NC(=O)CCCC(=O)NC23CNCCNCC(C)(CNCCNC2)CNCCNC3)C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N1)C(=O)O]
the formulation further comprising:
about 7 to about 13% (v/v) ethanol;
about 0.3 to about 1.2% (w/v) sodium chloride; and
about 0.02 to about 0.1% (w/v) gentisic acid, or a salt thereof;
wherein the formulation has a pH of between about 4 to about 8.

2. An aqueous formulation according to claim 1, wherein the formulation comprises:
about 10% (v/v) ethanol;
about 0.9% (w/v) sodium chloride;
about 0.06% (w/v) gentisic acid, or a salt thereof;
wherein the formulation comprises an acetate salt; and
wherein the formulation has a pH of about 6.0.

3. An aqueous formulation for parenteral administration comprising a compound of Formula (I), or a salt thereof, complexed with a Cu ion 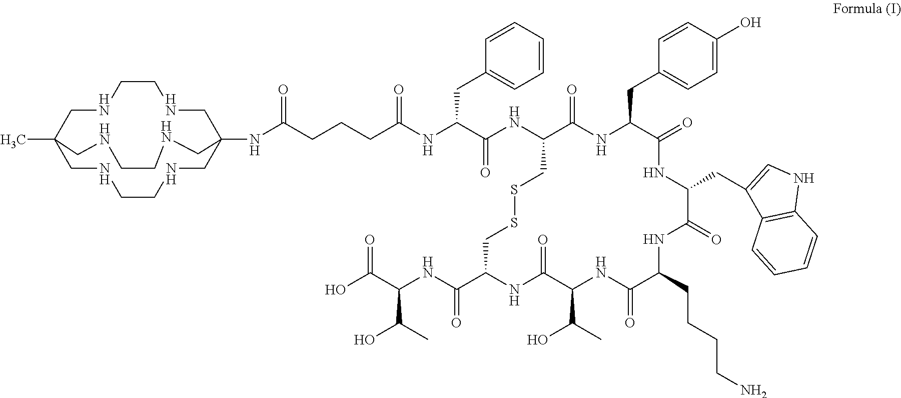 [SMILES: CC(O)[C@H](NC(=O)[C@@H]1CSSC[C@H](NC(=O)[C@@H](Cc2ccccc2)NC(=O)CCCC(=O)NC23CNCCNCC(C)(CNCCNC2)CNCCNC3)C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N1)C(=O)O]
the formulation further comprising:
about 7 to about 13% (v/v) ethanol;
about 0.3 to about 1.2% (w/v) sodium chloride;
about 0.02 to about 0.1% (w/v) gentisic acid, or a salt thereof; and
about 1.0 to about 4.0 mg/mL L-methionine, or a salt thereof;
wherein the formulation has a pH of between about 4 to about 8.

4. An aqueous formulation according to claim 3, wherein the formulation comprises:
about 10% (v/v) ethanol;
about 0.9% (w/v) sodium chloride;
about 0.06% (w/v) gentisic acid, or a salt thereof; and
about 2.5 mg/mL L-methionine, or a salt thereof;
wherein the formulation comprises an acetate salt; and
wherein the formulation has a pH of about 6.0.

5. An aqueous formulation according to claim 1, wherein the compound of Formula (I) is in the form of an acetate salt.

6. An aqueous formulation according to claim 1, wherein the formulation comprises an acetate salt as a buffering agent.

7. An aqueous formulation according to claim 1, wherein the gentisic acid salt is sodium gentisate.

8. An aqueous formulation according to claim 1, wherein the concentration of gentisic acid, or a salt thereof, is no more than 0.056% (w/v).

9. An aqueous formulation according to claim 1, wherein the Cu ion is a Cu radioisotope.

10. An aqueous formulation according to claim 9, wherein the Cu radioisotope is selected from the group consisting of ⁶⁰Cu, ⁶¹Cu, ⁶⁴Cu and ⁶⁷Cu.

11. A process for preparing an aqueous formulation according to claim 1, the method comprising the steps of:
i) preparing a buffering solution of an acetate salt, wherein the buffering solution further comprises ethanol and gentisic acid, or a salt thereof;
ii) dissolving a compound of Formula (I), or a salt thereof, in the buffering solution obtained from step i);
iii) adding a solution of a Cu ion to the solution obtained from step ii);
iv) filtering the solution obtained from step iii) on to a stationary phase; and
v) washing the stationary phase of step iv) with ethanol and saline;
to recover an aqueous formulation comprising a compound of Formula (I), or a salt thereof, complexed with a Cu ion.

12. A process for preparing an aqueous formulation according to claim 1, the method comprising the steps of:
i) preparing a buffering solution of an acetate salt, wherein the buffering solution further comprises ethanol and gentisic acid, or a salt thereof;
ii) dissolving a compound of Formula (I), or a salt thereof, in the buffering solution obtained from step i);
iii) adding a solution of a Cu ion to the solution obtained from step ii);
iv) filtering the solution obtained from step iii) on to a stationary phase; and
v) washing the stationary phase of step iv) with ethanol and saline into a solution of L-methionine;
to recover an aqueous formulation comprising a compound of Formula (I), or a salt thereof, complexed with a Cu ion.

13. A process according to claim 11, wherein the acetate salt of the buffering solution is ammonium acetate.

14. A process according to claim 11, wherein the concentration of the buffering solution of an acetate salt is about 0.1 mol/L.

15. A process according to claim 11, wherein the ethanol is present in the buffering solution at a concentration of about 4% to about 10% (v/v).

16. A process according to claim 11, wherein the buffering solution contains sodium gentisate.

17. A process according to claim 11, wherein the solution of a Cu ion is a solution in hydrochloric acid, wherein the concentration of the hydrochloric acid solution is about 0.01 to about 0.10 mol/L.

18-23. (canceled)

24. A kit for making an aqueous formulation for parenteral administration comprising a compound of Formula (I), or a salt thereof, complexed with a Cu ion, the kit comprising: 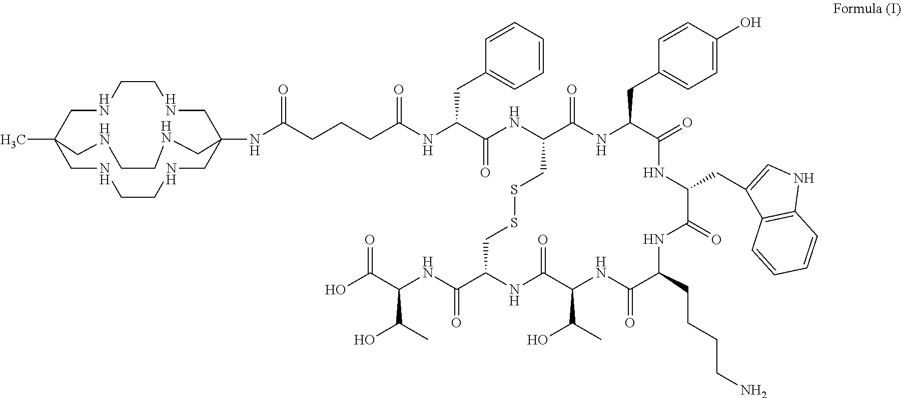 [SMILES: CC(O)[C@H](NC(=O)[C@@H]1CSSC[C@H](NC(=O)[C@@H](Cc2ccccc2)NC(=O)CCCC(=O)NC23CNCCNCC(C)(CNCCNC2)CNCCNC3)C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N1)C(=O)O]
a container comprising a lyophilised compound of Formula (I), or a salt thereof;
a container comprising a solution of a Cu ion; and instructions for preparing an aqueous formulation according to claim 1, including the addition of a buffered solution of ethanol, sodium chloride and gentisic acid, or a salt thereof.

25. A kit for making an aqueous formulation for parenteral administration comprising a compound of Formula (I) complexed with a Cu ion, or a salt thereof, the kit comprising: 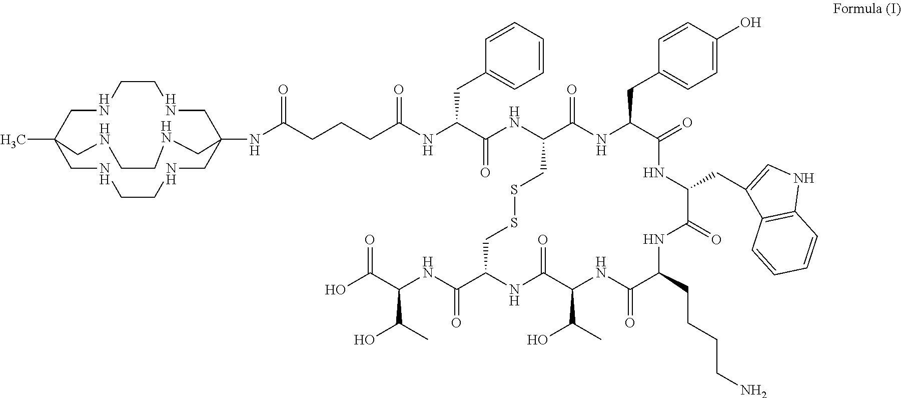 [SMILES: CC(O)[C@H](NC(=O)[C@@H]1CSSC[C@H](NC(=O)[C@@H](Cc2ccccc2)NC(=O)CCCC(=O)NC23CNCCNCC(C)(CNCCNC2)CNCCNC3)C(=O)N[C@@H](Cc2ccc(O)cc2)C(=O)N[C@H](Cc2c[nH]c3ccccc23)C(=O)N[C@@H](CCCCN)C(=O)N[C@@H](C(C)O)C(=O)N1)C(=O)O]
a container comprising a lyophilised compound of Formula (I), or a salt thereof;
a container comprising a solution of a Cu ion;
a container comprising a buffered solution of ethanol, sodium chloride and gentisic acid, or a salt thereof; and instructions for preparing an aqueous formulation according to claim 1, including the addition of a buffered solution of ethanol, sodium chloride and gentisic acid, or a salt thereof.

26. A kit according to claim 24, wherein the container comprising a buffered solution of ethanol, sodium chloride and gentisic acid further comprises L-methionine, or a salt thereof.

27. (canceled)
